(19) United States
(12) Patent Application Publication
(10) Pub. No.: US 2005/0042303 A1
(43) Pub. Date: Feb. 24, 2005

(54) Therapeutic protocols

(76) Inventors: Tracey Jean Brown, Flemington (AU); Richard Mark Fox, Melbourne (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/479,934

(22) PCT Filed: Aug. 27, 2002
(86) PCT No.: PCT/AU02/01160

(30) Foreign Application Priority Data
Aug. 27, 2001 (AU) PR 7302
Dec. 13, 2001 (AU) PR 9504

(51) Int. Cl. A61K 33/24; A61K 31/728

(52) U.S. Cl. 424/649; 514/54

(57) ABSTRACT

The present invention relates to the field of chemotherapy of diseases such as cell proliferation disorders including cancer. In particular, the present invention relates to the use of hyaluronan (HA) as a protective agent in the treatment of subjects. HA is administered in conjunction with a chemotherapeutic agent to facilitate the prolonged administration of a dose of the chemotherapeutic agent to be administered to a subject. Owing to the protective effects of the HA, the dose of chemotherapeutic agent may be substantially higher than a generally accepted effective dose, which would otherwise be expected to cause unacceptable side effects in the subject.

### FIELD OF THE INVENTION

The present invention relates to the field of chemotherapy of diseases such as cell proliferation disorders including cancer. In particular, the present invention relates to the use of hyaluronan (HA) as a protective agent in the treatment of subjects. HA is administered in conjunction with a chemotherapeutic agent to facilitate the prolonged administration of a dose of the chemotherapeutic agent to be administered to a subject. Owing to the protective effects of the HA, the dose of chemotherapeutic agent may be substantially higher than a generally accepted effective dose, which would otherwise be expected to cause unacceptable side effects in the subject.

### BACKGROUND OF THE INVENTION

Bibliographic details of references provided in the subject specification are listed at the end of the specification.

Reference to any prior art in this specification is not, and should not be taken as, an acknowledgment or any form of suggestion that this prior art forms part of the common general knowledge in any country.

Many diseases that afflict animals, including humans, are treated with chemotherapeutic agents. For example, chemotherapeutic agents have proven valuable in the treatment of neoplastic disorders including connective or autoimmune diseases, metabolic disorders, and dermatological diseases, and some of these agents are highly effective (e.g. vincristine and bleomycin) and do not cause any toxic side effects problems, such as neutropenia

Proper use of chemotherapeutic agents requires a thorough familiarity with the natural history and pathophysiology of the disease before selecting the chemotherapeutic agent, determining a dose, and undertaking therapy. Each subject must be carefully evaluated, with attention directed toward factors, which may potentiate toxicity, such as overt or occult infections, bleeding dyscrasias, poor nutritional status, and severe metabolic disturbances. In addition, the functional condition of certain major organs, such as liver, kidneys, and bone marrow, is extremely important. Therefore, the selection of the appropriate chemotherapeutic agent and devising an effective therapeutic regimen is influenced by the presentation of the subject. Unfortunately, many chemotherapeutics have severe side effects due to lack of selectivity between normal and malignant tissue.

Unwanted toxic side effects may include cardiac toxicity, hair loss, gastrointestinal toxicity (including nausea and vomiting), neurotoxicity, lung toxicity, asthma and bone marrow suppression (including neutropenia).

Bone marrow suppression associated with chemotherapy is the result, at least in part, of pronounced drug-induced depression of haematopoietic progenitor cells (HPCs) of the bone marrow (Shimamura et al., Exp. Hematol. 16: 681-685, 1988). The subsequent drop in feutrophil numbers leads to occurrences of secondary infections, the severity of which is directly related to the duration and severity of neutropenia (Bodey et al, Ann. Intern. Med. 64: 328, 1966). As a consequence of secondary infections patients are removed from their chemotherapy regime and placed on a treatment of broad-spectrum antibiotics resulting in limitations of the potential benefits of the cytotoxic treatment. However, despite the use of antibiotics, death from sepsis in severely neutropenic patients is not infrequent (Pettengel et al., Blood 80(6): 430-436, 1992).

The level of neutropenia is generally dependent on the regenerative capacity of the bone marrow and/or the dose of the drug being administered. Indeed, neutropenia is often the main reason for decisions to reduce the drug dose being given to a subject (Dotti et al., Haeinatologica 80: 142-145, 1995). Since drug dose reduction is typically accompanied by a loss of effectiveness, or potential effectiveness, of the chemotherapy, drug dose reduction is undesirable. Therefore, there is a need to identify administration regimes, or co-administered agents, which may lessen the incidence or severity of neutropenia associated with chemotherapy, thereby allowing drug dose reduction to be avoided or even enabling the possibility of using higher than standard doses of a chemotherapeutic agent.

In some early studies to reduce bone marrow suppression in a mouse model, accelerated recovery from neutropenia, by daily sub-cutaneous injections of human recombinant granulocyte colony-stimulating factor (rG-CSF) following initial injection of 5-fluorouracil (Shimamura et al., Blood 69: 353-355, 1987) and, more specifically, doxorubicin (Shimamura et al., 1988, supra), has been observed. Human rG-CSF exerts this effect by stimulating proliferation and differentiation of haematopoietic progenitor cells (Cohen et al., Proc. Natl. Acad. Sci. USA 84: 2484-2488, 1987). More recently, rG-CSF has been used as an adjunct, in patients undergoing cytotoxic chemotherapy, to enhance neutropenia recovery (Sheridan et al., Lancet 339: 640-644, 1992; Pettengell et al., 1992, supra; Anglin et al., Leuk. Lymphoma 11: 469-472, 1993; Kotaka et al., Int. J Urol. 6: 61-67, 1999). The application of rG-CSF, however, has to be optimally combined with repeated cycles of chemotherapy, due to the potential for the increased number of haematopoietic progenitor cells to become hypersensitive to cytotoxic drugs (Dotti et al., 1995, supra).

There is a need to develop protocols where a high cancer or target cell-killing dose of chemotherapeutic agent can be used without or with reduced levels of toxic side effects.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the determination that hyaluronan (HA) may be used as a protective agent in subjects, when these subjects receive treatment with a generally cytotoxic drug. Situations wherein subjects may receive a cytotoxic drug include the treatment of life-threatening diseases such as cancer. In these circumstances, the primary therapeutic objective is to effect the regression of malignant cells. However, the presently available therapeutically effective agents are less specific than would be preferred, and their administration results, eventually, in the death of many of the subject's normal cells as well. Where cancer regression is not effected quickly enough, the concomitant unwanted effect on normal cells may be so high that the subject's condition deteriorates to the point where treatment must be curtailed or stopped. This can have disastrous consequences for the subject undergoing treatment.

An agent that could moderate the need for cessation of treatment, for a period sufficiently long to facilitate the prolonged and increased therapeutic effect of the chemotherapeutic agent, would be a distinct advantage in the treatment of many forms of cancer and other diseases. While some of the beneficial effects of the inclusion of HA in treatment regimes have been known for some time, the protective effect of HA has not been appreciated until now.

Accordingly, in one aspect, the present invention provides a method which facilitates the prolonged administration of a dose of chemotherapeutic agent to a subject, wherein said a single dose may be up to 200% higher and/or the cumulative dose may be up to 600% higher than a generally accepted effective dose, said method comprising the pre- and/or co-administration of an effective amount of HA.

Under this regime, the pre- or co-administration of HA has the effect of ameliorating or even abolishing the otherwise unwanted concomitant deleterious effects on normal cells. As the patient's health is not being as adversely affected by the cytotoxic effects of the therapeutic agent, the said therapeutic agent may be administered at a higher than normal dose and allowed to act over a longer period of time. This increases the chances that the desirable cytotoxic effects of the administered drug, against unwanted malignant cells, will result in successful treatment.

Accordingly, another aspect of the present invention contemplates a method for the prolonged treatment of a subject with a dose of a chemotherapeutic agent where a single dose may be up to 200% higher and/or the cumulative dose may be up to 600% higher than a generally accepted effective dose, said method comprising pre- and/or co-administering an effective amount of HA with said chemotherapeutic agent.

The result of such treatment regimes is that the unacceptably severe side effects that are usually observed are obviated.

As HA is a polymeric molecule, it may be formulated to comprise molecules of varying molecular weights. Although lower molecular weight formulations are also effective in the methods of the present invention, preferred formulations comprise HA having a modal molecular weight in the range 750,000 to 2 million Da. Higher molecular weight HA has the advantage of a tertiary structure whereby, at low concentrations, it self-aggregates forming a three-dimensional meshwork. This meshwork exhibits the characteristics of controllable porosity and molecular dimension, which enables the establishment of equilibrium between therapeutic molecules held within the volumetric domain of the polysaccharide and the external environment. The “loading” of the HA three-dimensional structure with therapeutic molecules results in a controlled release of the therapeutic agent at the pathological site, subsequently overcoming non-specific targeting of healthy tissue. A particularly preferred weight range is 750,000-1,000,000 Da.

In a preferred embodiment, HA formulations may be administered to a subject simultaneously with or prior to administration of the chemotherapeutic agent. HA formulations may be generated in any number of ways, well known to those skilled in the art, including injectable solutions, powder formulations, tablets pills or capsules, or in any other convenient form.

### BRIEF DESCRIPTION OF THE FIGURES

FIGS. 1A, 1B and 1C are graphical representations showing the effect of doxorubicin on peripheral blood neutrophils in mice injected with doxorubicin and HyDox formulation. Male F1 mice aged between 11-13 weeks were injected with the above mentioned doxorubicin combinations at dosages of (A) 6 mg/kg, (B) 9 mg/kg and (C) 12 mg/kg, respectively. Blood was collected on Days 0 (baseline), 1, 4, 7, 10, and 14 after iv administration. Samples were analyzed for neutrophil numbers as outlined in materials and methods. Data for each day were graphed as average percentage of neutrophils of Day 0±SEM. (Broken line: Doxorubicin; solid line: HyDox; dotted line: HA). Experimental data can be found in Tables 1A and 1C.

FIGS. 2A, 2B, 2C, 2D and 2E are graphical representations showing the effect of doxorubicin on peripheral blood neutrophils in mice injected with doxorubicin and HA before doxorubicin. Male F1 mice, aged between 11-13 weeks, were injected with the above-mentioned drug combinations of dosages (A) 6 mg/kg, (B) 9 mg/kg, (C) 12 mg/kg, (D) 16 mg/kg and (E) 24 mg/kg doxorubicin, respectively. Blood was collected and analyzed for neutrophil numbers as previously described. Data for each day were graphed as the average percentage neutrophils of Day 0±SEM (broken line: Doxorubicin; solid line: HA injected 30 minutes before doxorubicin). Experimental data can be found in Tables 1A and 1B.

FIG. 3 is a graphical representation showing the percentage survival of mice injected with doxorubicin. In only two treatment groups were there any deaths before the end of the duration of this study. The treatment groups were 9 mg/kg (solid line) and 24 mg/kg (broken line) doxorubicin. All other mice, including those receiving the lower dosages of doxorubicin and the higher dosages in combination with hyaluronan, survived until the end of the study.

FIG. 4 is a graphical representation showing nadir neutropenia observed with escalating dosages of doxorubicin, with and without HA. For each treatment dosage, the lowest level of neutrophil counts was noted and graphed. Neutropenia is shown as a percentage of the day 0 measurement for each individual treatment group (solid line=HyDox; interrupted line=Dox; dotted line=HA before Dox)

FIGS. 5A, 5B, 5C, 5D and 5E are graphical representations showing the effect of doxorubicin on metabolic stress in mice injected with doxorubicin and HA before doxorubicin. Male F1 mice were injected with the above-mentioned combinations of doxorubicin, at dosages of (A) 6 mg/kg, (B) 9 mg/kg, (C) 12 mg/kg, (D) 16 mg/kg and (E) 24 mg/kg, respectively, and their weights were recorded on a daily basis. Data represent the loss or gain in weight, as average percentage of the original body weight ±SEM (broken line: Doxorubicin; solid line: HA before doxorubicin; dotted line:: HA). Data for graphs can be found in Tables 2A and 2B.

FIGS. 6A, 6B and 6C are graphical representations showing the effect of HyDox formulation on metabolic stress. Male F1 mice were intravenously injected with (A) 6 mg/kg, (B) 9 mg/kg and (C) 12 mg/kg doxorubicin, respectively, and equivalent dosages in the HyDox formulation. Body weights were recorded on a daily basis and graphed in the same manner as described in FIG. 3. Data represent mean ±SEM (broken line: Doxorubicin; solid line: HyDox; dotted line: HA). Data for these graphs can be found in Table 3.

FIG. 7 is a graphical representation showing the effect of HA/Dox on cardiotoxicity.

FIGS. 8A and 8B are photographic representations of electron micrographs showing changes in cardiac myocytes in rats after chronic exposure to (A) doxorubicin and (B) hyaluronan and doxorubicin.

FIGS. 9A and 9B are graphical representations showing the effect of doxorubicin on peripheral blood neutrophils in mice injected with HyDox. Mice were injected with HyDox with doxorubicin dosages of (A) 12 and (B) 16 mg/kg, respectively. The results were established by staggering the results of six groups, each bled every 4 days. In this manner, a daily assessment on the neutrophil content could be determined. Data plotted at each time point represent the average neutrophil count expressed as a percentage of the original day 0 count for that group (n=8) ±SEM. (solid line: HYDOX; broken line: Doxorubicin; dotted line: HA).

FIGS. 10A and 10B are graphical representations showing the effects of HyDox formulation on metabolic stress and food consumption. (A) Male F1 mice were intravenously injected with 12 and 16 mg/kg Dox and HyDox. Body weights were recorded on a daily basis and graphed, and the data presented as the loss or gain in weight as the percentage of the original starting body mass. Each data point is the average of 8 mice ±SEM. (B) In the same experimental groups, the food consumption was monitored on a daily basis and expressed as average mass of food eaten per day per mouse. Points represent the average mass of food where n=6-8. (broken line and ●: doxorubicin; solid line and +: HyDox), for panel A (dotted line): HA control.

FIGS. 11A, 11B, 11C and 11D are photographic representations of electron micrographs showing changes in the myocardium in rats after chronic exposure to doxorubicin ±hyaluronan. Rats received weekly injections of doxorubicin or HyDox over a 12 week period. The cumulative dose of doxorubicin was 13 mg/kg. Note large coalscent vacuoles (solid arrows) contained in myocytes in samples obtained from doxorubicin only (C) and HyDox (D)). The severity of myocyte vacuolation was much less evident in rats receiving HyDox (D). Myocyte vacuolation was least evident in control groups tested: no treatment (A), and hyaluronan (B).

FIGS. 12A, 12B, 12C and 12D are photographic representations of electron micrographs showing loss of myofibrillar mass in myocardium after chronic exposure to doxorubicin. Rats receiving doxorubicin (A) displayed a greater degree of myofibrillar lysis which was indicated by loss of characteristc parallel orientation of myofibrils and blurring of the Z-bands (dotted arrows). This loss was not as evident in samples from HyDox treated rats where ordered myofibril arays were still present (B, dashed arrows.) Mitochondria profiles also appeared enlarged and disruption of cristae was also noted. Severe disruption to mitochondrial function was evidenced by the appearance of myelin figures (A and B, solid arrows). These observations were not as frequent in rats receiving HyDox (B). In samples obtained from control groups, all contained intact mitochondria and an ordered array of myofibrild as indicated by the parallel alignment of the Z-bands (C and D:hyaluronan and no treatment respectively).

FIG. 13 is a graphical representation showing percentage of vacuolated myocytes as a score of cardiomyopathy. Sections of the intraventricular septum from rats were processed for transmission electron analysis and the degree of damage was scored. Because vacuolation of myocytes was a constant feature in all affected cells, this feature was used as an efficient way of assessing injury. On any one ultra thin section, the number of injured cells (i.e. vacuolated) was counted, as well as the number of unaffected cardiac myocytes, provided that the complete transection could be assessed. The proportion of injured cells in each sample was then expressed as a percentage. Data represent the mean ±SEM (n=8-10). HA=hyaluronan; NT=no treatment; * p<0.001 (t-test) when compared with Dox only group.

FIGS. 14A, 14B, 14C and 14D are graphical representations showing antioxidant levels after chronic exposure to doxorubicin ±HA. The activities of the antioxidant enzymes Catalase (CAT) [A]; Glutathione peroxidase [C]; superoxide dismutase (SOD) [D], and levels of reduced glutathione [B] were measured after chronic exposure to Dox and HyDox. Each bar represents the average estimation of 8-10 animals ±SEM. In [B], the liver data have been removed and re-graphed (1: insert) so that cardiac GSH content can be seen. * p<0.05 (one-way analysis of variance) between treatment groups [A], and between HyDox and Dox [B].

FIG. 15A is a graphical representation showing the effects of CMF/HA therapy on body weight in mice. Pre- or co-administration of HA with CMF resulted in statistically significant increase in body weight. Six treatment groups are shown circle=saline; inverted triangle=HA on days 1, 2; square=HA on days 1, 3; diamond=CMF on days 1, 2; triangle=CMF/HA on days 1, 2; hexagon=HA on days 1, 3, followed by CMF. Further details are provided in Table 12.

FIG. 15B is a graphical representation showing linear regression analysis of the effects of CMF/HA therapy on mouse body mass.

FIG. 16 is a graphical representation showing the effect of the CMF/HA treatment regime on mouse mean survival. Co-administration of HA eliminated toxicity and resulted in a statistically significant (p<0.05) increase in the survival period. Further details are provided in Table 18.

FIG. 17 is a graphical representation of a dose-response curve showing the effect of a range of different concentrations of HA on the cytotoxicity of Dox—at different concentrations—on H9C2 differentiated cardiomyocytes.

### DETAILED DESCRIPTION OF THE PREFERRED EMBODIMENTS

In accordance with the present invention, it has been determined that, in the presence of an effective amount of HA, undesirable and cumulative cytotoxic effects of chemotherapeutic agents on normal cells are ameliorated. Usually, this unwanted cytotoxicity eventually causes the need to remove the chemotherapeutic agent or, at least, reduce the levels or duration of treatment.

According to the methods of the present invention, however, HA can be used to avoid the need for undesirable reductions in the dose or duration of administration of a chemotherapeutic agent, in chemotherapy patients. Unacceptably severe side effects including neutropenia, cardiac toxicity and gastro-intestinal toxicity as manifested by nausea, vomiting and severe weight loss, are mitigated against or avoided. That is, by pre- and/or co-administering an effective amount of HA, the dose of a chemotherapeutic agent may be increased to a level above that which would be possible if the said chemotherapeutic agent were administered in the absence of the effective amount of HA.

Accordingly, in one aspect, the present invention contemplates a method which facilitates the prolonged administration of a dose of chemotherapeutic agent to a subject, wherein said dose may be up to a single dose may be up to 200% higher and/or the cumulative dose may be up to 600% higher than a generally accepted effective dose, said method comprising the pre- and/or co-administration of an effective amount of HA.

Persons skilled in the art can readily determine if or when a subject receiving chemotherapy is suffering from unacceptable side effects and should be subjected to a variation or alteration of the treatment. Where, for example, a chemotherapy patient were receiving a cytotoxic chemotherapeutic agent such as 5-fluorouracil and/or doxorubicin and, over time, would be expected to exhibit unacceptable side effects such as neutropenia, cardiac toxicity, and gastrointestinal toxicity, instead of reducing the dose of the chemotherapeutic agent, the medical practitioner may, in accordance with the present invention, maintain or increase the dose of the chemotherapeutic agent by pre- or co-administering an effective amount of HA. Such side effects may also be referred to as “unacceptably severe side effects”.

By “pre- or co-administering”, it is to be understood that the HA may be administered before, at the same time as, or after the subject has been administered the chemotherapeutic agent. Where the HA is administered before the chemotherapeutic agent, preferably it is administered from about 24 hours to about 5 minutes, more preferably from about 12 hours to about 10 minutes, still more preferably from about 5 hours to about 10 minutes, and most preferably about 0.5 hour, before administration of the chemotherapeutic agent. Where the HA is administered after the chemotherapeutic agent, preferably it is administered 0.05 to 24 hr, more preferably 0.1 to 5 hr, and most preferably about 0.5 hr, after administration of the chemotherapeutic agent.

Preferably, the chemotherapeutic agent that is administered is selected from the group consisting of carmustine (BCNU), chlorambucil (Leukeran), cisplatin (Platinol), Cytarabine, doxorubicin (Adriamycin), fluorouracil (5-FU), methotrexate (Mexate), Cyclophosphamide, CPT 11, etoposide, plicamycin (Mithracin) and taxanes such as, for example, paclitaxel.

Most preferably, the chemotherapeutic agent is doxorubicin (Adriamycin). Doxorubicin is an anthracycline glycoside antibiotic and is one of the most effective anti-neoplastic drugs used in clinical practice (Carter, J. Nat. Cancer Inst. 55: 1265-1274, 1975). Its clinical usefulness extends across a wide range of neoplastic conditions, such as solid tumors including breast, lung and thyroid carcinoma; hematologic malignancies such as acute leukemia and lymphoma and soft tissue and bone sarcoma and neuroblastoma.

In general therapeutic treatments, persons skilled in the art are able to exercise judgement, based on experience, concerning the point at which continued administration will result in unacceptable side effects. The continued administration of a cytotoxic agent beyond this point in time is referred to herein as “prolonged administration” of the drug.

Accordingly, another aspect of the present invention provides a method for the prolonged treatment of a subject with a dose of a chemotherapeutic agent which is up to 200% higher than a generally accepted effective dose, said method comprising pre- and/or co-administering an effective amount of HA with said chemotherapeutic agent.

The term “subject” as used herein refers to any animal having a disease or condition which is in need of treatment with a chemotherapeutic agent. Preferably, the subject is suffering from a cellular proliferative disorder (e.g. a neoplastic disorder). Subjects for the purposes of the invention include, but are not limited to, mammals such as humans and other primates, livestock animals (e.g. sheep, horses, cows, pigs donkeys), laboratory test animals (e.g. rats mice, rabbits, guinea pigs) and companion animals (e.g. dogs and cats). Humans are the most preferred of the primates.

In the treatment of subjects such as patients suffering from neoplastic disease, there are “generally accepted effective doses” of cytotoxic drug, depending on the drug being administered, with which the treating medical practitioner would be familiar, and above which medical practitioners are at present generally unwilling to prescribe. This is because, as mentioned above, deleterious effects of prescribed cytotoxic drugs on normal cells accumulate over time until the treatment must be reduced or removed in order to prevent the continuation of these unacceptably severe side effects. For a range of different chemotherapeutic agents, the person skilled in the art would be aware of what constituted a generally accepted effective dose.

In accordance with the present invention, however, doses of a chemotherapeutic agent in a single dose may be up to 200% higher and/or the cumulative dose may be up to 600% higher may be administered. The expression “up to 200%” is to be understood to include and encompass any integer or fraction between approximately 5% to approximately 200%. Preferred ranges are from about 20% to about 150% higher. In a most preferred embodiment, the generally accepted effective dose is increased by from about 35% to about 100%.

Accordingly, another aspect of the present invention provides a method of treating a subject, said method comprising administering to said subject a dose of a chemotherapeutic agent which is higher than a dose which causes clinically unacceptable side effects wherein said chemotherapeutic agent is pre- and/or co-administered with an effective amount of HA.

As used herein, the term “effective amount” means an amount of HA which is effective to diminish the severity of the side effects of the chemotherapeutic agent, such that a dose of the chemotherapeutic agent which is equal to or higher than the generally accepted effective dose, may be used.

The specific “effective amount” will, obviously, vary with such factors as the identity of the pre- or co-administered chemotherapeutic agent, the physical condition of the patient, the type of mammal being treated, the duration of the treatment, and the nature of concurrent therapy (if any). However, generally, an effective amount of HA will be of the order of about 0.5 mg to about 10 mg per kilogram body weight, with a preferred amount being in the range of between about 5 mg to about 20 mg per kilogram body weight per day (from about 0.3 g to about 3 g per patient per day).

Being a polymeric molecule, HA molecules may exhibit a range of varying molecule weights. HA formulations may, therefore, comprise molecules of different molecular weights. Almost any average of modal molecular weight formulation of HA may be effective in the methods of the present invention. Preferred formulations, however, exhibit higher rather than lower modal molecular weights. HA having a modal molecular weight in the range 750,000 to 2 million Da is preferred. Higher molecular weight HA has the advantage of forming a tertiary structure whereby at low concentrations, it self-aggregates forming a three-dimensional meshwork, which exhibits the characteristics of controllable porosity and molecular dimension, which enables the establishment of equilibrium between therapeutic molecules held within the volumetric domain of the polysaccharide and the external environment. The “loading” of the HA three-dimensional structure with therapeutic molecules results in a controlled release of the therapeutic agent at the pathological site, subsequently overcoming non-specific targeting of healthy tissue. A particularly preferred weight range is 750,000-1,000,000 Da.

The methods of the present invention enable the continued or prolonged use of a dose of a chemotherapeutic agent at or above that which would be expected to cause unacceptably severe side effects in the subject, if said chemotherapeutic agent were administered in the absence of the effective amount of HA. The dosage amount will vary, depending upon the identity of the particular chemotherapeutic agent and other factors. However, by way of example, the methods of the present invention allow use of doses of doxorubicin of ≧60 mg/m², more preferably ≧80 mg/m² and most preferably, ≧100 mg/m².

The chemotherapeutic agent and/or HA may be administered orally, topically, parenterally, by intra-tumoral injection, or in dosage unit formulations containing conventional non-toxic pharmaceutically acceptable carriers, adjuvants, and vehicles. The term “parenteral” as used herein includes subcutaneous injections, aerosol, intravenous, intramuscular, intrathecal, intracranial injection, sub-lingual, lymphatically, or infusion or perfusion techniques.

As used herein, a “pharmaceutically acceptable carrier” is a pharmaceutically acceptable solvent, suspending agent or vehicle for delivering the chemotherapeutic agent and/or HA to the subject. The carrier may be liquid or solid and is selected with the planned manner of administration in mind.

Accordingly, the chemotherapeutic agent and/or HA may be formulated for use in the methods of the present invention as, for example, topical, oral, and parenteral pharmaceutical formulations. Therefore the chemotherapeutic agent and/or HA may be administered orally as tablets, aqueous or oily suspensions, lozenges, troches, powders, granules, emulsions, capsules, syrups or elixirs. Formulations for oral use may contain one or more of the following: sweetening agents, flavouring agents, colouring agents and preserving agents. Tablet formulations of the chemotherapeutic agent and/or HA may contain the active ingredients in admixture with non-toxic pharmaceutically acceptable excipients which are suitable for the manufacture of tablets. These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. Such tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in U.S. Pat. Nos. 4,256,108, 4,160,452; and 4,265,874 to form osmotic therapeutic tablets for controlled release.

The chemotherapeutic agent as well as the HA can be administered for use in the methods of the present invention by in vivo application, parenterally by injection or by gradual perfusion over time independently or together. Administration route may be intravenous, intra-arterial, intra-peritoneal, intramuscular, subcutaneous, intra-cavity, or trans-dermal. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

HA may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

It is envisioned that the methods of the present invention can be used where a subject is to be treated with a chemotherapeutic agent for the treatment of a cell proliferative disorder. By “cell proliferative disorder” is meant that a cell or cells demonstrate abnormal growth, typically aberrant growth, leading to a neoplasm, tumor or a cancer. Cell proliferative disorders include, for example, cancers of the breast, lung, prostate, kidney, skin, neural tissue, ovary, uterus, liver, pancreas, epithelium, head and neck tissue as well of the gastric, intestinal, exocrine, endocrine, lymphatic or haematopoietic system.

The methods of the present invention may also be used where the subject is to be treated with a chemotherapeutic agent for the treatment of a neurodegenerative disorders, hormonal imbalance and the like.

Any discussion of documents, acts, materials, devices, articles or the like, which has been included in the present specification, is solely for the purpose of providing a context for the present invention. It is not to be taken as an admission that any or all of these matters either formed part of the prior art base or were common general kiowledge in the field relevant to the present invention as it existed in Australia or elsewhere before the priority date of each claim of this application.

The invention will now be further described by way of reference to the following non-limiting examples. It should be understood, however, that the examples following are illustrative only, and should not be taken in any way as a restriction on the generality of the invention described herein. In particular, while the invention is described in detail in relation to cancer, it will be clearly understood that the findings herein are not limited to treatment of cancer. For example, HA may be used for treatment of the cytotoxic side effects of chemotherapeutic agents used to treat other conditions.

### EXAMPLE 1

### In vivo Model of Cytotoxicity (I)

Drugs for Intravenous Injection

The anti-cancer drug, doxornbicin (Adriamycin), hereinafter referred to as “Dox”, was purchased from Asta Medical (NSW, Australia) in syringes, each containing 2.5 mL at a final concentration of 2 mg/mL or as doxorubicin hydrochloride powder which was reconstituted in 0.9% sterile sodium chloride to a final concentration of 2 mg/mL. Desiccated hyaluronan (HA), 824,000 KDa, was purchased from Pearce Pharmaceuticals (Victoria, Australia) and was dissolved in sterile water to a final concentration of 10 mg/mL, filter sterilized through a 0.22 μm filter, and stored at 4° C. until used. Hyaluronan mixed with doxorubicin (hereinafter referred to as “HyDox”) was prepared by mixing calculated volumes of 10 mg/mL hyaluronan with a calculated volume 0.5 mg/mL Dox with to achieve the desired dosages of doxorubicin. The dosage of HA used throughout this study was 13.3 mg/kg of bodyweight. The dosages of Dox studied were, 6, 9, 12, 16 and 24 mg/kg.

Experimental Animals

Male F1 mice (C57×CBA; 11-13 weeks old), used for in vivo studies, were obtained from The Animal Central Division (Monash University, Victoria, Australia). The mice were distributed at random into each treatment group (6 per treatment group) and allowed to acclimatize for 2-3 weeks before commencing the study. The treatment groups were split into ‘drug only’ which received the dosages 6, 9, 12, 16, and 24 mg/kg of Dox, respectively: HA (13.3 mg/kg) injected 30 minutes before 6, 9, 12, 16, and 24 mg/kg dox: and 6, 9 and 12 mg/kg HyDox. Two control groups received intravenous injections of physiological saline, HA (13.3 mg/kg) respectively. Before a treatment group was started, blood (200 μL) from each experimental animal was collected by retroorbital sinus bleed and immediately transferred to tube containing EDTA, gently flick mixed and stored on ice until analysis. This was taken as day 0, after which the mice were given a single bolus intravenous injection via tail vein with corresponding control, drug, or HA/drug combinations. The mice were then sequentially bled on days 1, 4, 7, 10 and 14 by the aforementioned method. All blood samples were analysed for white blood cell composition in an Adiva 120 Differential Coulter Counter (Bayer Diagnostics, USA). Throughout the duration of the study, mouse mass, food intake and general activity levels were recorded on a daily basis. On day 14, mice were sacrificed by cervical dislocation and the heart, lungs, liver, spleen, kidneys, stomach, intestines and testicles removed and immediately fixed in 10% v/v formalin in phosphate buffered saline.

General Toxicity

Mice that received dox, in either the HyDox formulation or pre-sensitized with 13.3 mg/kg HA 30 minutes before the Dox injection, survived the duration of the experiment. Only in the Dox treatment groups at higher drug dosages were mice fatalities observed. In the 24 mg/kg Dox group, 2 mice survived until the end of the study, whereas mice receiving HA 30 minutes prior to the same dosage of dox, survived the treatment period (see FIG. 3). With the exception of one mouse in the 9 mg/kg Dox group, which died unexpectedly at day 8 after iv administration of drug, all other mice survived until the end of the study.

Haematolozcal Determinations

Neutropenia

The average neutrophil number for day 0 for all groups was 1.39×10³ cells/μL blood ±0.60 SD (min value=0.54, max value=3.34×10³ cells/μL blood). To normalize the data to allow comparison between treatment groups, experimental data were expressed as the percentage of the baseline bleed for each individual mouse and subsequent measurements on day 1, 4, 7, 10 and 14 (where day 0 bleed=100%).

6 mg/kg Doxorubicin and HyDox Treatment Groups

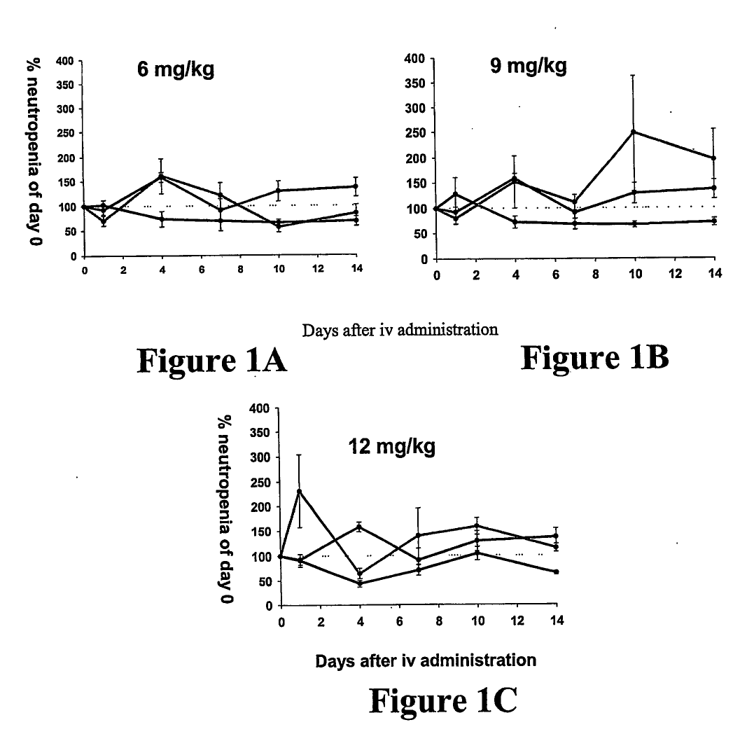

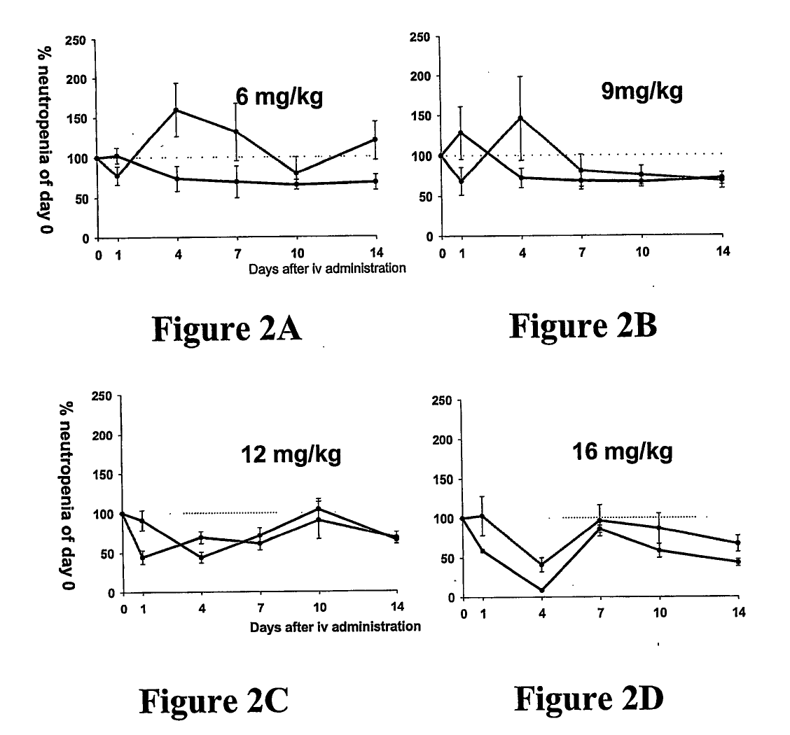

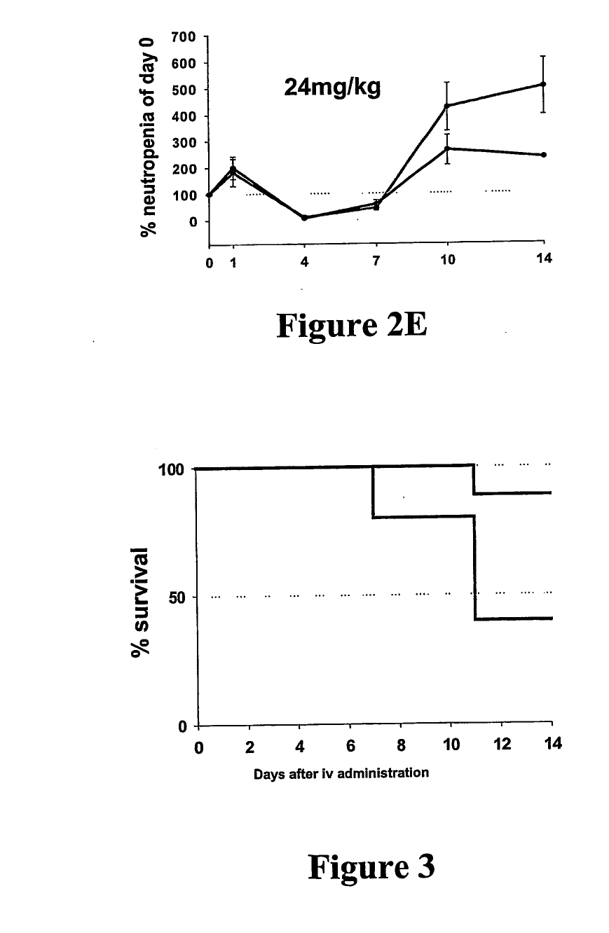

The lowest dosage of Dox in this study was 6 mg/kg, which is equivalent to a human dosage of 30 mg/m². In both drug combinations (HyDox or HA before dox), neutropenia was actually reversed and an increase in the level of peripheral neutrophils was observed when compared with the “Dox only” treatment group (FIGS. 1 and 2, panels labeled 6 mg/kg). The maximum value was approximately 160% (see Table 1A, B and C) and was observed at day 4. This gradually declined by day 7, but still remained higher than the “Dox only” group, whose neutrophil level never recovered to baseline even by day 14.

TABLE 1A
Neutropenia data expressed as percentage of Day 0 for all doxorubicin dosages
and drug combinations: neutropenia data for doxorubicin injections

| | Treatment Groups: Doxorubicin only | | | | |
|---|---|---|---|---|---|
| | 6 mg/kg Dox | 9 mg/kg Dox | 12 mg/kg Dox | 16 mg/kg Dox | 24 mg/kg Dox |
| Day | (n = 12) | (n = 9) | (n = 10) | (n = 3) | (n = 5) |
| 0 | 100 | 100 | 100 | 100 | 100 |
| 1 | 102.4 ± 9.3 | 128.1 ± 32.7 | 91 ± 12.5 | 59.1 ± 2.5 | 199.5 ± 42.5 |
| 4 | 73.7 ± 15.9 | 72.4 ± 12 | 44.1 ± 6.9 | 8.2 ± 1.5 | 6.7 ± 3.6 |
| 7 | 69.4 ± 20 | 68.5 ± 10.6 | 71.1 ± 10.3 | 85.9 ± 4.7 | 58.9 ± 14.7 |
| 10 | 65.6 ± 9.6 | 67.10 ± 6.2 | 104 ± 13.2 | 58.5 ± 8.7 | 262.7 ± 56.6 |
| 14 | 68.3 ± 9.6 | 71.4 ± 7.7 | 65 ± 4 | 43.1 ± 4.6 | 233* |

*At this end point of the experiment, only 2 mice remained. due to one of these samples unexpectedly clotting only one blood sample was analysed for neutrophil content.

TABLE 1B
Neutropenia data for Hyaluronan (13.3 mg/Kg, 824,000
Da) injected 30 minutes before doxorubicin

| | Treatment Groups: HA injected 30 minutes before Dox | | | | |
|---|---|---|---|---|---|
| | 6 mg/kg Dox | 9 mg/kg Dox | 12 mg/kg Dox | 16 mg/kg Dox | 24 mg/kg Dox |
| Day | (n = 5) | (n = 6) | (n = 5) | (n = 6) | (n = 5) |
| 0 | 100 | 100 | 100 | 100 | 100 |
| 1 | 78 ± 11.4 | 68.70 ± 17.3 | 44.7 ± 8.5 | 102.8 ± 24.8 | 189.4 ± 40.4 |
| 4 | 159.4 ± 33.9 | 146.2 ± 52.3 | 69.1 ± 7.6 | 41.1 ± 8.9 | 10 ± 5.8 |
| 7 | 131.4 ± 36.1 | 81.3 ± 19.9 | 61.4 ± 8.2 | 96.2 ± 19.6 | 56.3 ± 13.7 |
| 10 | 79.8 ± 20.2 | 75.5 ± 11.9 | 90.2 ± 23.4 | 86.5 ± 18.9 | 424.6 ± 65 |
| 14 | 119.4 ± 23.8 | 68.3 ± 9.8 | 67.6 ± 7.5 | 67 ± 10.1 | 501.2 ± 105.5 |

TABLE 1C
Neutropenia data for HyDox formulation and control injections

| | Treatment groups: HyDox formulation | | | Controls | |
|---|---|---|---|---|---|
| | 6 mg/kg | 9 mg/kg | 12 mg/kg | Hyaluronan | Saline |
| | HyDox | HyDox | HyDox | 133 mg/kg | (400 μL) |
| Day | (n = 6) | (n = 6) | (n = 5) | (n = 6) | (n = 4) |
| 0 | 100 | 100 | 100 | 100 | 100 |
| 1 | 70 ± 10.2 | 80 ± 10.6 | 230.8 ± 73.3 | 92.6 ± 10.3 | 78.8 ± 11.7 |
| 4 | 161 ± 36 | 151.9 ± 51.6 | 64.7 ± 10.7 | 158 ± 10.1 | 111 ± 8.4 |
| 7 | 121.2 ± 25.4 | 111.3 ± 14.8 | 139.1 ± 56.5 | 91.1 ± 23.3 | 66.6 ± 6 |
| 10 | 56.7 ± 10.8 | 247.7 ± 114.6 | 158.6 ± 17.2 | 128.8 ± 20.1 | 78.9 ± 12.7 |
| 14 | 84.5 ± 17.3 | 195.6 ± 58.5 | 114.6 ± 7.7 | 136.2 ± 18.6 | 105.2 ± 6.5 |

9 mg/kg Doxorubicin and HyDox Treatment Groups

Dox at 9 mg/kg is equivalent to a human dose of 45 mg/m² and a similar trend was again observed in both the HyDox formulation and when HA was administered before Dox (FIGS. 1 and 2, respectively). In both these treatment groups, however, there was a slight decrease in the percentage of peripheral neutrophils, but by day 4 these values had increased to approximately 150%. In the “Dox only” group, drug-induced (nadir) neutropenia was observed at day 4 and was approximately 72% of day 0 baseline bleeds. Subsequent measurements on day 7, 10 and 14 revealed the percentage of neutrophils in the HA before Dox retuning to the pattern observed in the Dox only group (FIG. 2, panel labeled 9 mg/kg).

12 mg/kg Doxorubicin and HyDox Treatment Groups

The protective effect of HA against neutropenia was even more evident in the 12 mg/kg HyDox dosage (60 mg/m², human equivalent) when compared with the HA before Dox group (FIGS. 1 and 2, respectively). Only on day 4 did the peripheral neutrophils from 12 mg/kg HyDox group drop to an average level of 64%. Thereafter, they recovered and maintained a value greater than the baseline bleeds. Again, the lowest level of neutropenia was observed in the Dox only group (FIG. 1, panel 12 mg/kg, black line).

16 mg/kg and 24 mg/kg Doxorubicin and HyDox Treatment Groups

The 16 mg/kg and 24 mg/kg Dox groups did not include HyDox due to the difficulties encountered with its formulation and volume restrictions for injection into a mouse. Therefore, at these dosages, only Dox, and HA before Dox, were studied. By day 1, the level of neutrophils in the 16 mg/kg Dox (80 mg/m² human equivalent dose) was approximately 59% while a value of 103% was observed where HA was administered prior to drug (FIG. 2; panel labelled 16 mg/kg). The lowest level of neutropenia was observed at day 4, and was more severe in the “Dox only” group. The degree of neutropenia was most severe in the 24 mg/kg Dox dose, and was equally severe whether administered with or without HA (FIG. 2; panel labeled 24 mg/kg).

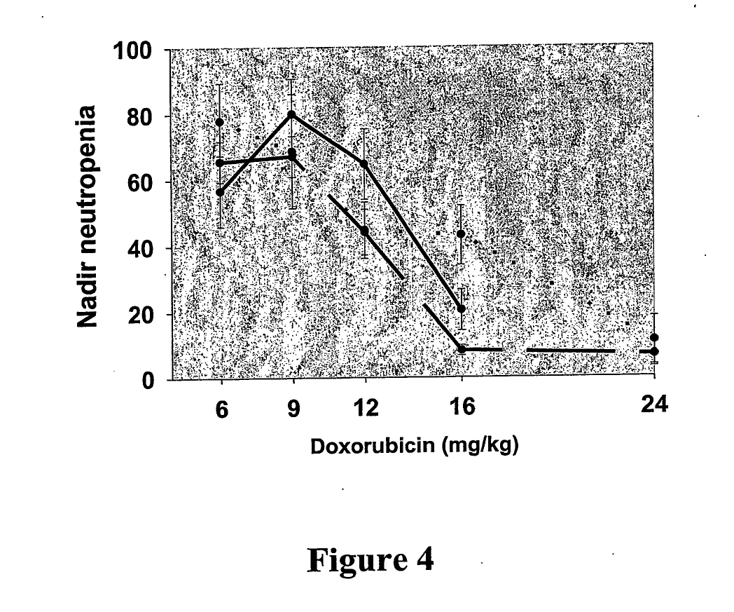

Throughout all the treatment groups, the nadir neutropenia was dose-dependent and was more severe in treatment groups receiving Dox only, compared with equivalent Dox dosages administered as either HyDox or HA before Dox (see FIG. 4). The administration of control injection of saline did not have any considerable effect on the level of neutrophil levels and was therefore not graphed. The data are presented in Table 1c.

Quite unexpected was the result obtained from the HA control group, which was injected at the same dosage of all other experimental groups (13.3 mg/kg). The percentage of peripheral neutrophils in the HA group closely resembled the 6 and 9 mg/kg dosages in both the HyDox formulation (FIG. 1) and where HA was administered 30 minutes before drug (FIG. 2).

Other Haematological Measurements

As well as neutrophil determination, the red blood cell, percentage hemaglobin, percentage hematocrit, percentage platelets and lymphocytes were also considered. Data for these parameters can be found in Tables 2 to 6. All data presented in these Tables are expressed as a percentage of the pre-bleed value, obtained from day 0 prior to injection of drug.

TABLE 2
Percent hemoglobin

| | Treatment Groups | | | | | | | |
|---|---|---|---|---|---|---|---|---|
| | | HA b/f | | | | | | |
| | 6 mg/kg | 6 mg/kg | 6 mg/kg | 9 mg/kg | HA b/f 9 | 9 mg/kg | 12 mg/kg | HA b/f |
| | Dox | Dox | HyDOX | Dox | mg/kg Dox | HyDox | Dox | 12 mg/kg |
| Day | (n = 12) | (n = 5) | (n = 6) | (n = 9) | (n = 6) | (n = 6) | (n = 10) | Dox (n = 5) |
| 0 | 100 | 100 | 100 | 100 | 100 | 100 | 100 | 100 |
| 1 | 83.9 ± 1.90 | 82.3 ± 2.40 | 84.7 ± 1.80 | 85.8 ± 2.10 | 80.8 ± 4.50 | 78.8 ± 6.90 | 79.7 ± 1.50 | 75.7 ± 3.20 |
| 4 | 67.3 ± 2.10 | 66.5 ± 3.10 | 69.0 ± 1.10 | 68.20 ± 3.80 | 70.1 ± 5.50 | 67.7 ± 1.80 | 60.3 ± 2.30 | 58.8 ± 2.60 |
| 7 | 57.0 ± 1.30 | 77.4 ± 2.10 | 79.7 ± 2.70 | 45.7 ± 4.60 | 76.0 ± 7.10 | 59.6 ± 1.10 | 47.7 ± 1.90 | 59.5 ± 1.80 |
| 10 | 81.4 ± 1.14 | 91.2 ± 1.30 | 95.7 ± 2.50 | 70.7 ± 3.10 | 91.7 ± 2.20 | 98.3 ± 1.80 | 70.1 ± 4.20 | 93.1 ± 0.80 |
| 14 | 100 ± 1.30 | 104.7 ± 1.70 | 103 ± 3.00 | 98.5 ± 1.50 | 102.6 ± 3.50 | 85.4 ± 2.10 | 90.6 ± 1.30 | 93.10 ± 2.20 |

Treatment Groups

| | | | | | HA b/f | | |
|---|---|---|---|---|---|---|---|
| | 12 mg/kg | 16 mg/kg | HA b/f | 24 mg/kg | 24 mg/kg | HA (13.3 | |
| | HyDOX | Dox | 16 mg/kg | Dox | Dox | mg/kg) | Saline |
| Day | (n = 5) | (n = 3) | Dox (n = 6) | (n = 5) | (n = 5) | (n = 6) | (n = 6) |
| 0 | 100 | 100 | 100 | 100 | 100 | 100 | 100 |
| 1 | 72.9 ± 6.20 | ** | 86.5 ± 4.0 | 82.6 ± 1.60 | 81.7 ± 2.30 | 88.8 ± 1.5 | 81.8 ± 1.3 |
| 4 | 62.2 ± 2.10 | 74.10 ± 2.40 | 74.2 ± 4.20 | 68.9 ± 3.20 | 76.3 ± 2.60 | 81.1 ± 1.1 | 79.1 ± 2.2 |
| 7 | 63.80 ± 13.0 | 51.4 ± 4.60 | 52.8 ± 3.50 | 63.2 ± 6.70 | 61.3 ± 3.60 | 88.3 ± 3.5 | 85.2 ± 0.9 |
| 10 | 52.4 ± 5.00 | 64.3 ± 3.40 | 80.7 ± 3.80 | 42.2 ± 3.40 | 43.1 ± 2.50 | 89.10 ± 3.3 | 85.0 ± 1.9 |
| 14 | 93.0 ± 2.20 | 86.0 ± 3.60 | 101.2 ± 4.0 | 79.3 | 65.5 ± 2.90 | 95.3 ± 1.9 | 92.6 ± 1.3 |

(b/f) = indicates hyaluronan injection 30 minutes before doxorubicin.
** = samples from this treatment group read ‘zero’ on day 1 of measurement.

TABLE 3
Percentage hematocrit

| | Treatment Group | | | | | | | |
|---|---|---|---|---|---|---|---|---|
| | | HA b/f | | | HA b/f | | | |
| | 6 mg/kg | 6 mg/kg | 6 mg/kg | 9 mg/kg | 9 mg/kg | 9 mg/kg | 12 mg/kg | HA b/f |
| | Dox | Dox | HyDOX | Dox | Dox | (n = 6) | Dox | 12 mg/kg |
| Day | (n = 12) | (n = 5) | (n = 6) | (n = 9) | (n = 6) | HyDox | (n = 10) | Dox (n = 5) |
| 0 | 100 | 100 | 100 | 100 | 100 | 100 | 100 | 100 |
| 1 | 95.5 ± 11.50 | 180.1 ± 91.20 | 82.6 ± 1.40 | 93.4 ± 6.90 | 43.2 ± 14.20 | 96.4 ± 6.40 | 94.8 ± 1.50 | 92.3 ± 7.20 |
| 4 | 75.3 ± 8.80 | 275.6 ± 128.30 | 54.8 ± 9.30 | 90.2 ± 9.80 | 132.7 ± 66.60 | 82.30 ± 5.80 | 52.9 ± 1.80 | 68.6 ± 5.40 |
| 7 | 70.1 ± 8.80 | 204.7 ± 107.0 | 74.5 ± 5.10 | 74.9 ± 10.10 | 163.2 ± 84.70 | 75.0 ± 4.80 | 50.5 ± 4.30 | 73.1 ± 5.40 |
| 10 | 103.7 ± 10.60 | 106.5 ± 75.4 | 99.4 ± 3.0 | 92.10 ± 4.45 | 31.7 ± 13.20 | 107.5 ± 3.10 | 74.2 ± 5.10 | 96.0 ± 6.70 |
| 14 | 121.4 ± 12.7 | 483.9 ± 239.00 | 108.0 ± 2.80 | 98.8 | 220.5 ± 92.70 | 106.5 ± 4.40 | 60.0 ± 6.40 | 59.2 ± 7.30 |

Treatment Group

| | | | | | HA b/f | | |
|---|---|---|---|---|---|---|---|
| | 12 mg/kg | | HA b/f | 24 mg/kg | 2 mg/kg | HA (13.3 | |
| | HyDOX | 16 mg/kg | 16 mg/kg | Dox | Dox | mg/kg) | Saline |
| Day | (n = 5) | Dox (n = 3) | Dox (n = 6) | (n = 5) | (n = 5) | (n = 6) | (n = 6) |
| 0 | 100 | 100 | 100 | 100 | 100 | 100 | 100 |
| 1 | 77.8 ± 5.60 | 110.7 ± 21.5 | 102.8 ± 24.80 | 80.4 ± 2.50 | 77.7 ± 2.30 | 85.7 ± 1.9 | 78.8 ± 11.7 |
| 4 | 54.0 ± 2.50 | 88.0 ± 17.7 | 41.10 ± 8.90 | 34.6 ± 8.0 | 31.6 ± 9.20 | 83.0 ± 1.6 | 111 ± 8.4 |
| 7 | 46.6 ± 3.30 | 25.20 ± 3.20 | 96.2 ± 19.60 | 59.2 ± 4.80 | 55.9 ± 3.50 | 89.8 ± 2.3 | 66.6 ± 6 |
| 10 | 50.9 ± 5.80 | 90.7 ± 16.70 | 86.5 ± 18.90 | 37.5 ± 3.20 | 37.3 ± 1.80 | 50.9 ± 5.8 | 78.9 ± 12.7 |
| 14 | 84.0 ± 3.40 | 107.8 ± 18.90 | 67.0 ± 10.10 | 85.7 | 73.3 ± 2.80 | 87.3 ± 5.2 | 105.2 ± 6.5 |

TABLE 4
Percentage platelets

| | Treatment Groups | | | | | | | |
|---|---|---|---|---|---|---|---|---|
| | | HA b/f | | | HA b/f | | | HA b/f |
| | 6 mg/kg | 6 g/kg | 6 mg/kg | 9 mg/kg | 9 mg/kg | 9 mg/kg | 12 mg/kg | 12 mg/kg |
| | Dox | Dox | HyDOX | Dox | Dox | HyDox | Dox | Dox |
| Day | (n = 12) | (n = 5) | (n = 6) | (n = 9) | (n = 6) | (n = 6) | (n = 10) | (n = 5) |
| 0 | 100 | 100 | 100 | 100 | 100 | 100 | 100 | 100 |
| 1 | 98.0 ± 11.0 | 100.4 ± 6.60 | 102.0 ± 6.60 | 72.6 ± 5.30 | 105.7 ± 7.90 | 76.4 ± 2.90 | 105.2 ± 6.0 | 115.7 ± 31.40 |
| 4 | 124.7 ± 18.0 | 151.9 ± 8.50 | 192.2 ± 10.40 | 103.8 ± 10.10 | 154.3 ± 10.3 | 107.9 ± 8.30 | 150.1 ± 12.6 | 195.6 ± 54.40 |
| 7 | 175.1 ± 17.2 | 155.7 ± 8.30 | 136.2 ± 11.20 | 152.7 ± 15.0 | 171.7 ± 10.10 | 135.2 ± 14.80 | 205.6 ± 10.0 | 204.7 ± 25.60 |
| 10 | 98.1 ± 6.60 | 86.6 ± 3.90 | 91.1 ± 2.50 | 112.1 ± 12.2 | 97.1 ± 7.60 | 51.0 ± 7.90 | 190.4 ± 15.4 | 88.0 ± 13.0 |
| 14 | 84.7 ± 10.5 | 75.0 ± 5.50 | 94.7 ± 5.80 | 96.0 ± 7.20 | 71.1 ± 4.80 | 58.10 ± 2.80 | 147.0 ± 9.80 | 158.1 ± 31.0 |

Treatment Groups

| | | | HA b/f | | HA b/f | | |
|---|---|---|---|---|---|---|---|
| | 12 mg/kg | 16 mg/kg | 16 mg/kg | 24 mg/kg | 24 mg/kg | HA | |
| | HyDOX | Dox | Dox | Dox | Dox | (13.3 mg/kg) | Saline |
| Day | (n = 5) | (n = 3) | (n = 6) | (n = 5) | (n = 5) | (n = 6) | (n = 6) |
| 0 | 100 | 100 | 100 | 100 | 100 | 100 | 100 |
| 1 | 76.9 ± 4.50 | 106.6 ± 23.6 | 71.7 ± 1.20 | 78.4 ± 13.10 | 86.8 ± 9.10 | 100.7 ± 10.2 | 104 ± 1.9 |
| 4 | 114.60 ± 10.90 | 159.6 ± 24.3 | 103.5 ± 5.10 | 91.2 ± 6.00 | 81.8 ± 12.80 | 135.6 ± 8.6 | 123.4 ± 9.8 |
| 7 | 187.8 ± 15.20 | 145.3 ± 6.70 | 123.9 ± 8.10 | 59.4 ± 11.40 | 58.30 ± 4.80 | 138.9 ± 14.5 | 125 ± 9.4 |
| 10 | 228.3 ± 43.60 | 223.1 ± 13.3 | 107.3 ± 15.5 | 254.9 ± 48.30 | 324.5 ± 32.5 | 112.2 ± 11.6 | 134.2 ± 12.6 |
| 14 | 127.3 ± 29.2 | 137.9 ± 6.20 | 91.1 ± 11.20 | 218.20 | 222.5 ± 18.40 | 110.6 ± 10.4 | 94.7 ± 19.9 |

TABLE 5
Percentage red blood cells

| | Treatment Groups | | | | | | | |
|---|---|---|---|---|---|---|---|---|
| | | HA b/f | | | HA b/f | | | HA b/f |
| | 6 mg/kg | 6 mg/kg | 6 mg/kg | 9 mg/kg | 9 mg/kg | 9 mg/kg | 12 mg/kg | 12 mg/kg |
| | Dox | Dox | HyDOX | Dox | Dox | HyDox | Dox | Dox |
| Day | (n = 12) | (n = 5) | (n = 6) | (n = 9) | (n = 6) | (n = 6) | (n = 10) | (n = 5) |
| 0 | 100 | 100 | 100 | 100 | 100 | 100 | 100 | 100 |
| 1 | 124.6 ± 17.4 | 78.0 ± 11.40 | 85.8 ± 1.10 | 98.6 ± 7.20 | 44.0 ± 13.20 | 102.7 ± 6.90 | 77.0 ± 1.20 | 97.8 ± 15.30 |
| 4 | 89.5 ± 10.30 | 159.4 ± 33.90 | 58.5 ± 3.30 | 98.5 ± 10.90 | 134.7 ± 60.3 | 90.7 ± 6.40 | 55.4 ± 1.70 | 73.6 ± 11.80 |
| 7 | 80.20 ± 10.10 | 131.4 ± 36.10 | 67.5 ± 4.50 | 80.4 ± 13.10 | 136.2 ± 61.2 | 77.1 ± 5.0 | 49.1 ± 3.10 | 67.3 ± 9.10 |
| 10 | 106.3 ± 14.4 | 79.8 ± 20.20 | 88.4 ± 2.40 | 92.90 | 26.4 ± 8.90 | 102.4 ± 12.40 | 61.0 ± 4.60 | 77.0 ± 10.10 |
| 14 | 128.8 ± 17.0 | 119.4 ± 23.8 | 96.1 ± 2.10 | 103.50 | 185.1 ± 69.6 | 104.3 ± 6.10 | 59.4 ± 9.30 | 50.9 ± 5.80 |

Treatment Groups

| | | | HA b/f | | | | |
|---|---|---|---|---|---|---|---|
| | 12 mg/kg | 16 mg/kg | 16 mg/kg | 24 mg/kg | HA b/f | HA (13.3 | |
| | HyDOX | Dox | Dox | Dox | 24 mg/kg | mg/kg) | Saline |
| Day | (n = 5) | (n = 3) | (n = 6) | (n = 5) | Dox (n = 5) | (n = 6) | (n = 6) |
| 0 | 100 | 100 | 100 | 100 | 100 | 100 | 100 |
| 1 | 77.3 ± 2.60 | 113.7 ± 23.30 | 102.8 ± 24.80 | 84.6 ± 1.90 | 81.6 ± 2.90 | 83.4 ± 2.1 | 80.5 ± 2.0 |
| 4 | 56.6 ± 2.50 | 90.10 ± 41.10 | 41.1 ± 8.90 | 35.5 ± 7.20 | 32.10 ± 9.20 | 73.4 ± 1.5 | 72.4 ± 1.8 |
| 7 | 48.2 ± 2.70 | 25.40 ± 3.50 | 96.2 ± 19.60 | 64.3 ± 6.50 | 61.9 ± 3.90 | 77.2 ± 1.9 | 76.8 ± 2.8 |
| 10 | 46.2 ± 4.90 | 72.7 ± 13.8 | 86.5 ± 18.90 | 41.6 ± 4.10 | 41.4 ± 2.30 | 44.0 ± 4.9 | 42.6 ± 2.8 |
| 14 | 76.2 ± 3.60 | 96.6 ± 18.0 | 67.0 ± 10.10 | 64.50 | 59.9 ± 2.10 | 76.3 ± 5.1 | 78.9 ± 4.1 |

TABLE 6
Percentage lymphocytes

| | Treatment Group | | | | | | | |
|---|---|---|---|---|---|---|---|---|
| | | HA b/f | | | | | | |
| | 6 mg/kg | 6 mg/kg | 6 mg/kg | 9 mg/kg | HA b/f | 9 mg/kg | 12 mg/kg | HA b/f |
| | Dox | Dox | HyDOX | Dox | 9 mg/kg | HyDox | Dox | 12 mg/kg |
| Day | (n = 12) | (n = 5) | (n = 6) | (n = 9) | Dox (n = 6) | (n = 6) | (n = 10) | Dox (n = 5) |
| 0 | 100 | 100 | 100 | 100 | 100 | 100 | 100 | 100 |
| 1 | 122.1 ± 14.70 | 77.5 ± 11.10 | 142.1 ± 23.90 | 70.5 ± 8.40 | 59.0 ± 6.10 | 65.6 ± 8.30 | 36.8 ± 5.10 | 38.2 ± 3.50 |
| 4 | 71.5 ± 5.30 | 92.6 ± 7.40 | 134.9 ± 29.7 | 49.70 ± 4.60 | 114.5 ± 20.0 | 65.6 ± 11.30 | 39.8 ± 6.60 | 47.7 ± 4.90 |
| 7 | 97.1 ± 9.50 | 89.6 ± 21.9 | 122.4 ± 15.30 | 95.3 ± 13.7 | 122.3 ± 10.20 | 119.2 ± 11.40 | 59.7 ± 6.80 | 68.2 ± 4.90 |
| 10 | 84.1 ± 6.60 | 80.2 ± 21.8 | 138.4 ± 11.9 | 100.4 ± 12.9 | 68.9 ± 14.80 | 63.20 ± 7.60 | 85.5 ± 5.80 | 52.4 ± 5.80 |
| 14 | 77.9 ± 7.80 | 91.2 ± 17.5 | 126.4 ± 11.5 | 82.60 ± 8.10 | 89.1 ± 10.50 | 88.7 ± 8.50 | 51.2 ± 5.40 | 60.4 ± 2.00 |

Treatment Group

| | 12 mg/kg | 16 mg/kg | HA b/f | | HA b/f | HA (13.3 | |
|---|---|---|---|---|---|---|---|
| | HyDOX | Dox | 16 mg/kg | 24 mg/kg | 24 mg/kg | mg/kg) | Saline |
| Day | (n = 5) | (n = 3) | Dox (n = 6) | Dox (n = 5) | Dox (n = 5) | n = 6 | (n = 6) |
| 0 | 100 | 100 | 100 | 100 | 100 | 100 | 100 |
| 1 | 41.0 ± 9.60 | 119.1 ± 16.0 | 144.1 ± 18.0 | 48.1 ± 6.80 | 60.1 ± 11.90 | 101.2 ± 6.7 | 78.8 ± 11.7 |
| 4 | 57.5 ± 11.70 | 35.3 ± 10.70 | 69.6 ± 13.40 | 17.0 ± 3.30 | 19.9 ± 6.70 | 168.4 ± 11.8 | 111 ± 8.4 |
| 7 | 86.3 ± 14.40 | 64.5 ± 4.80 | 99.6 ± 13.70 | 32.4 ± 6.40 | 43.3 ± 8.10 | 90.9 ± 9.6 | 66.6 ± 6 |
| 10 | 183.7 ± 46.4 | 111.7 ± 11.50 | 108.2 ± 4.70 | 66.8 ± 17.4 | 110.5 ± 16.10 | 108 ± 13.4 | 78.9 ± 12.7 |
| 14 | 74.5 ± 13.90 | 71.4 ± 12.10 | 90.7 ± 27.4 | 112.2 | 120.9 ± 13.20 | 119.1 ± 17.5 | 105.2 ± 6.5 |

In all dosages, a greater percentage of peripheral lymphocytes was observed in the HyDox formulation when compared with the Dox equivalent (see Table 6). The HA control injection increased peripheral lymphocytes, with maximum of 168% increase observed at day 4. The increase in platelets in the 24 mg/kg Dox dosages correlated to the increase in neutrophil numbers at the same dosage, perhaps indicating internal tissue damage and infection.

Metabolic Stress

All treatment groups were monitored for weight loss on a daily basis. At the end of the study the data were expressed as percentage of original body mass and graphed in this manner. The percentage of weight lost in both control groups of HA and saline injections was similar. Therefore, only HA data were plotted with experimental data for comparison. Weight loss data for all experimental groups, including HA and saline controls, can be found in Tables 7A, 7B and 8.

TABLE 7A
Weight loss data for Doxorubicin and HA before Doxorubin injections
for 6, 9 and 24 mg/kg dosages. Data are expressed as a percentage
of original body mass prior to commencement of treatment

| | Treatment groups | | | | | |
|---|---|---|---|---|---|---|
| | | HA before 6 | | HA before 9 | 24 mg/kg | HA before 24 |
| | 6 mg/kg Dox | mg/kg Dox | 9 mg/kg Dox | mg/kg Dox | Dox | mg/kg Dox |
| Day | (n = 6) | (n = 5) | (n = 6) | (n = 6) | (n = 4) | (n = 5) |
| 0 | 0.0 | 0.0 | 0.0 | 0.0 | 0.0 | 0.0 |
| 1 | −1.10 ± 0.65 | −2.70 ± 0.40 | −0.90 ± 0.73 | −0.70 ± 0.50 | −3.60 ± 0.40 | −3.90 ± 1.00 |
| 2 | −1.00 ± 0.73 | −4.00 ± 0.90 | −1.50 ± 0.61 | −1.30 ± 0.40 | −6.40 ± 0.35 | −6.80 ± 1.10 |
| 3 | −3.00 ± 0.40 | −4.40 ± 0.80 | −5.00 ± 0.16 | −1.10 ± 0.80 | −12.60 ± 0.90 | −13.80 ± 0.90 |
| 4 | −5.90 ± 0.73 | −4.20 ± 0.50 | −7.60 ± 0.69 | −1.70 ± 0.90 | −15.90 ± 0.80 | −17.50 ± 1.00 |
| 7 | −4.50 ± 0.53 | −8.60 ± 0.70 | −9.70 ± 0.89 | −5.20 ± 1.70 | −26.20 ± 2.10 | −27.50 ± 0.50 |
| 8 | −2.70 ± 1.55 | −5.90 ± 0.60 | −8.30 ± 0.61 | −2.70 ± 1.70 | −24.90 ± 1.75 | −25.80 ± 0.35 |
| 9 | −2.10 ± 0.97 | −3.00 ± 0.50 | −6.30 ± 0.73 | −0.40 ± 1.20 | −24.80 ± 2.30 | −24.10 ± 0.40 |
| 10 | −5.00 ± 0.89 | −4.20 ± 0.60 | −8.00 ± 0.81 | −1.60 ± 1.10 | −26.30 ± 2.30 | −23.90 ± 0.85 |
| 11 | −2.90 ± 1.10 | −5.60 ± 0.30 | −6.90 ± 0.85 | −3.00 ± 1.20 | −27.70 ± 2.75 | −23.60 ± 0.70 |
| 14 | −1.90 ± 0.85 | −5.10 ± 0.71 | −6.30 ± 0.48 | −2.91 ± 0.44 | −16.80 ± 2.50 | −22.10 ± 1.80 |

TABLE 7B
Weight loss data for Doxorubicin and HA before Doxorubicin
injections for 12 and 16 mg/kg dosages

| | | HA before 12 | | HA before 16 |
|---|---|---|---|---|
| | 12 mg/kg Dox | mg/kg Dox | 16 mg/kg Dox | mg/kg Dox |
| Day | (n = 4) | (n = 5) | (n = 3) | (n = 6) |
| 0 | 0.0 | 0.0 | 0.0 | 0.0 |
| 1 | −1.90 ± 0.75 | −2.10 ± 0.89 | −5.60 ± 0.60 | −4.60 ± 0.69 |
| 4 | −6.10 ± 0.85 | −2.40 ± 0.84 | −14.50 ± 0.90 | −10.90 ± 1.60 |
| 5 | −6.80 ± 0.95 | −4.10 ± 0.84 | −15.20 ± 1.70 | −11.20 ± 2.00 |
| 6 | −7.80 ± 1.10 | −4.30 ± 1.43 | −11.90 ± 2.40 | −11.50 ± 1.80 |
| 7 | −9.50 ± 1.10 | −4.50 ± 1.65 | −14.20 ± 1.80 | −11.80 ± 0.90 |
| 8 | −9.70 ± 1.75 | −3.90 ± 2.54 | −16.80 ± 1.70 | −12.60 ± 0.60 |
| 11 | −12.70 ± 1.35 | −4.10 ± 2.50 | −12.40 ± 1.67 | −9.00 ± 0.65 |
| 12 | −9.10 ± 1.45 | −5.00 ± 2.19 | −10.50 ± 1.67 | −7.20 ± 0.69 |
| 13 | −7.70 ± 1.75 | −2.50 ± 2.45 | −9.10 ± 1.32 | −5.90 ± 0.89 |
| 14 | −10.80 ± 1.70 | 4.80 ± 2.28 | −9.80 ± 1.03 | −7.80 ± 1.02 |

TABLE 8
Weight-loss data for HyDox injections
for 6, 9 and 12 mg/kg dosages

| | Treatment Groups | | |
|---|---|---|---|
| | 6 mg/kg | 6 mg/kg | 9 mg/kg |
| | Dox | HyDox | Dox |
| Day | (n = 6) | (n = 6) | (n = 3) |
| 0 | 0.0 | 0.0 | 0.0 |
| 1 | −1.40 ± 0.93 | 0.50 ± 1.02 | −2.30 ± 0.51 |
| 2 | −1.50 ± 1.30 | 0.90 ± 1.10 | |
| 3 | −4.60 ± 1.38 | 0.10 ± 0.97 | |
| 4 | −7.30 ± 2.32 | 0.70 ± 1.06 | −6.60 ± 2.36 |
| 5 | | | −8.70 ± 4.07 |
| 6 | | | −7.50 ± 5.88 |
| 7 | −4.50 ± 2.12 | 0.90 ± 1.06 | −8.30 ± 4.67 |
| 8 | −1.40 ± 1.95 | 3.50 ± 1.26 | −7.10 ± 6.50 |
| 9 | 2.30 ± 2.04 | 7.10 ± 1.42 | |
| 10 | −0.70 ± 1.75 | 5.10 ± 1.38 | |
| 11 | 1.50 ± 1.30 | 6.40 ± 1.42 | 0.20 ± 0.63 |
| 12 | | | 0.40 ± 0.23 |
| 13 | | | 3.80 ± 1.38 |
| 14 | 0.60 ± 1.47 | 5.50 ± 1.30 | 3.10 ± 1.32 |

| | Treatment Groups | | |
|---|---|---|---|
| | 9 mg/kg | 12 mg/kg | 12 mg/kg |
| | HyDox | Dox | HyDox |
| Day | (n = 6) | (n = 6) | (n = 5) |
| 0 | 0.0 | 0.0 | 0.0 |
| 1 | −0.80 ± 0.85 | −4.90 ± 0.65 | −2.80 ± 0.70 |
| 2 | | −5.90 ± 0.57 | −4.00 ± 0.80 |
| 3 | | −7.70 ± 0.65 | −7.60 ± 0.70 |
| 4 | 0.20 ± 1.14 | −9.00 ± 0.53 | −8.70 ± 0.60 |
| 5 | −0.80 ± 1.42 | | |
| 6 | −1.00 ± 0.85 | | |
| 7 | 1.60 ± 1.91 | −11.50 ± 1.14 | −9.80 ± 0.90 |
| 8 | 5.20 ± 2.08 | −12.20 ± 1.10 | −10.80 ± 1.10 |
| 9 | | −11.10 ± 1.26 | −9.70 ± 0.70 |
| 10 | | −12.70 ± 1.30 | −10.70 ± 0.90 |
| 11 | −2.50 ± 1.87 | −13.50 ± 1.74 | −11.00 ± 0.60 |
| 12 | 6.20 ± 2.69 | | |
| 13 | 5.50 ± 3.30 | | |
| 14 | 0.90 ± 2.77 | −11.80 ± 1.63 | −10.80 ± 0.70 |

Controls

| Day | HA (13.3 mg/kg) N = 6 | Saline N = 6 |
|---|---|---|
| 0 | 0.0 | 0.0 |
| 1 | −2.5 ± 1.5 | −3.2 ± 0.48 |
| 2 | −3.0 ± 1.4 | −1.9 ± 0.97 |
| 3 | −3.2 ± 1.3 | −4.10 ± 1.02 |
| 4 | −4.0 ± 1.2 | −5.10 ± 1.95 |
| 7 | −4.8 ± 1.0 | −4.0 ± 0.94 |
| 8 | −5.7 ± 1.1 | −4.7 ± 1.14 |
| 9 | −4.8 ± 1.5 | −4.1 ± 1.22 |
| 10 | −7.1 ± 1.3 | −5.9 ± 1.26 |
| 11 | −6.6 ± 1.2 | −4.4 ± 1.34 |
| 14 | −4.6 ± 1.3 | −2.9 ± 0.89 |

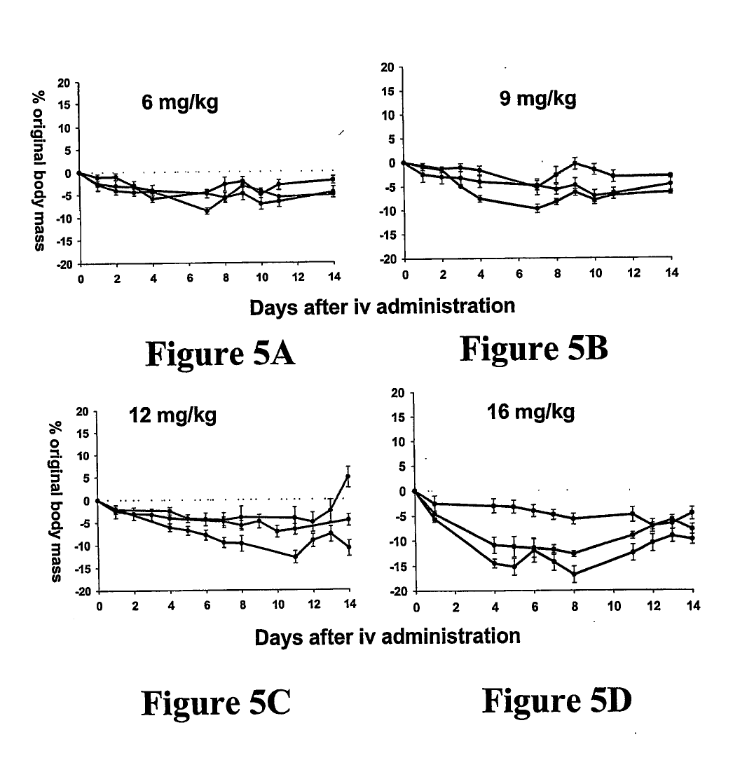

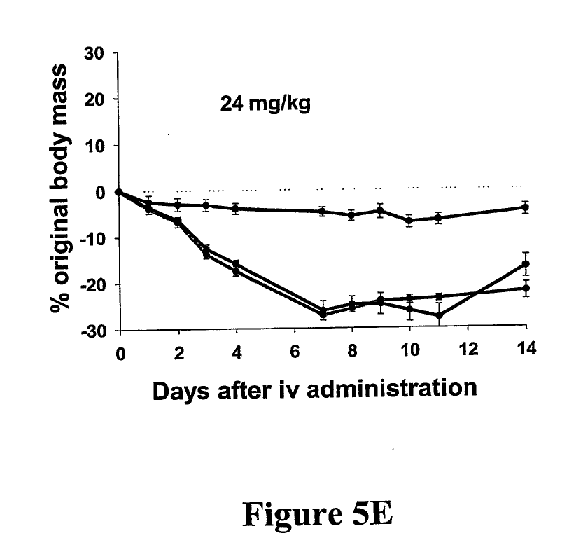

The weight lost by the mice was dose dependant: the highest degree of loss was observed in the 24 mg/kg dosage group, where almost 25% of original weight was lost by day 7 (FIG. 5; panel labeled 24 mg/kg). In the lower dosages of 6 and 9 mg/kg dox, the weight loss in HA before Dox was comparable with the HA control data and by day 8 in the 9 mg/kg HA before Dox started to re-gain close to original starting body mass. The 9 mg/kg p Dox only group lost a maximum of close to −10%, whereas the HA before Dox only lost a maximum of −5.7% (FIG. 5). In the HA before 12 mg/kg Dox dosage (60 mg/m² human equivalent) weight loss was again comparable to that observed in the control groups but when in the Dox only group the weight loss was starting to become more apparent with almost 13% of original body mass lost by day 11 (FIG. 5).

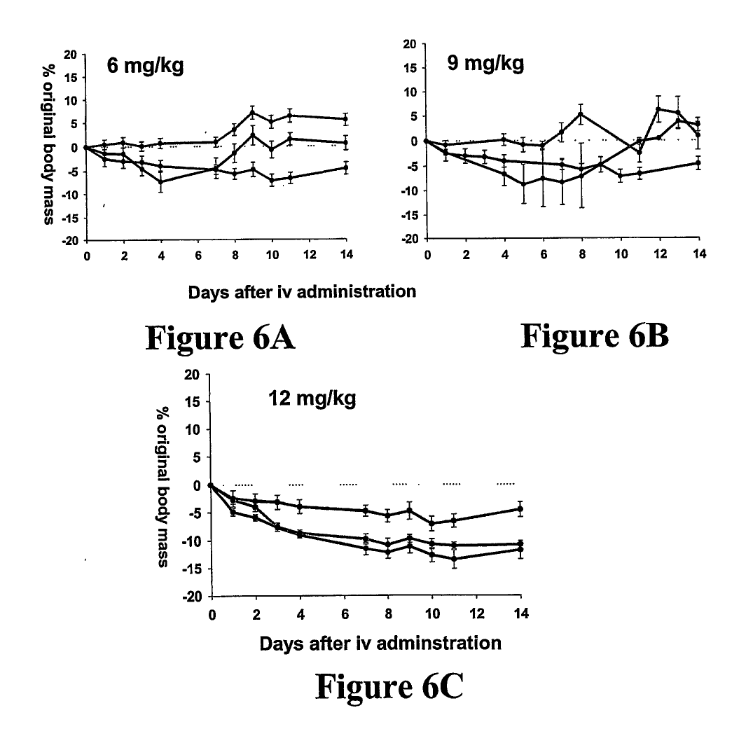

Interestingly when the equivalent dose was tested in the HyDox formulation the weight loss was equivalent to the corresponding Dox only group (FIG. 6). When the lower HyDox dosages were examined, however, the 6 and 9 mg/kg groups did not lose weight, and by day 7 actually started to gain weight above that of original starting body mass.

The above data indicate that HyDox, and HA administered 30 minutes before Dox have the capacity to reduce neutropenia and actually stimulate neutrophil release into blood circulation, at human equivalent dosages of 30, 45 and 60 mg/m². This is a significant finding in light of current chemotherapy regimes which are often hampered by non-completion of treatment due to drug related neutropenia

### EXAMPLE 2

### In vivo Model of Cardiotoxicity (I)

Experimental Animals

Adult female Wistar rats (10 weeks old) were purchased from The Animal Central Division (Monash University, Victoria, Australia) and were randomly divided into 6 experimental groups (n=8 per treatment group). Group 1 through to 3 received weekly intravenous injections of: (1) 1.5 mg/kg Dox only; (2) HA administered 30 minutes before 1.5 mg/kg Dox; and (3) 1.5 mg/kg HyDox. Group 4 through to 5 received weekly intravenous injections of comparable volumes of saline and 13.3 mg/kg HA, respectively. Group 6 received no treatment.

Prior to the commencement of the study, blood (500 μL) was collected from each rat via a tail vein bleed procedure. Blood was collected in 1.5 mL Capiject tube and allowed to clot for a minimum of 30 minutes. After this time, the clot was pelleted by centrifugation at 3,500 gₐᵥ for 2 minutes and the serum was transferred to a clean 1.5 mL Eppendorf and immediately stored at −20° C. until further analysis. After pre-bleeds, each experimental animal was injected with respective drug combination or control treatment and 24 hours after administration the rats were tail-bled and processed as described above. Thereafter, animals were injected with drug combinations at weekly intervals for 12 weeks, and blood was collected 24 hours after treatment. The total cumulative dose of Dox received by animals in Groups 1, 2 and 3 was 18 mg/kg.

Processing of Tissue for Analysis

One week after the last injection, the rats were killed by decapitation and internal organs were fixed in 10% v/v formalin in phosphate buffered saline. Prior to tissue fixation, portions of the kidney, liver, and skeletal muscle and heart were quickly removed and placed in tubes on ice. The tissue was either used immediately or stored at −80° C. until assayed. Each sample was minced into small pieces, washed vigorously in two changes of ice cold 0.25 M sucrose containing 1 mM EDTA, then washed in 50 mM potassium phosphate buffer pH 7.8 containing 0.1 mM EDTA prior to recording the tissues ‘wet’ weight. The tissue was then resuspended in 2 mL of ice-phosphate-EDTA buffer and homogenized on ice for three 5-second bursts over 5 minutes using an Ultraturrax homogenizer. The fine suspension was made up to a final volume of 4 mL with the phosphate-EDTA buffer and used for the enzyme and GSH assays described below.

Antioxidant Enzymes and GSH Assay

From the 4 mL tissue homogenate, 1 mL was transferred to a clean 1.5 mL Eppendorf and stored on ice. Another 1 mL of phosphate buffer was added to 3 mL tissue homogenate to make the final volume 4 mL. For the enzyme assays, the crude homogenate (4 mL) was centrifuged for 60 minutes at 100,000×g at 4° C. in a Beckman TL-100 ultracentrifuge fitted with a TLA100.2 rotor. After centrifugation, the clear high-speed supernatant was aliquoted and stored at −80° C. until direct determinations of glutathione peroxidase (GP), catalase, and superoxide dismutase (SOD) activities were made.

For the GSH assay, 0.25 mL of 25% w/v metaphosphoric acid was added to the 1 nL of the original crude homogenate, mixed thoroughly and incubated on ice for 15 minutes. The protein precipitate was pelleted by centrifugation at 10,000 g for 10 minutes at 4° C. The clear supernatant was transferred to a clean tube and either stored at −70° C. for no longer than three days or GSH levels were directly determined. The level of GSH was determined using a fluorometric assay using 0-phthalaldehyde as described by Cohn & Lyle (Anal. Biochem. 14(3): 434-440, 1966).

SOD activity was determined measuring the Cytochrome c by the method according to McCord & Fridovich (J. Biol. Chem. 244(22): 6056-6063, 1969). GP activity was assessed by an indirect coupled assay (Paglia & Valentine, J. Lab. Clin. Med. 70(1): 158-169, 1967). Catalase activity was measured by following the decomposition of H₂0₂ at 240 nm by a method described by Claiborne (Am. Rev. Respir. Dis. 131(6):947-949, 1985).

The protein concentrations of 100,000 g supernatants and 10,000 g supernatants were determined using the BCA protein assay.

Serum Levels of Cardiac Troponin-T (cTnT)

To quantify the degree of cardiotoxicity caused by Dox, the serum levels of cTnT were determined on each serum sample collected throughout the study using an immunoassay for cardiac troponin T from Roche Diagnostics (Victoria, Australia).

Analysis of Cardia Damage by Electron Microscopy

Immediately after animal sacrifice, a traverse section of the intraventricular septum was dissected from the heart and fixed in 2% glutaraldehyde, 2% paraformaldehyde and 4% glucose in phosphate buffer. Standard post-buffering, osmication and processing were followed. Specimens were examined with a Philip's electron microscope and the frequency and severity of cardiac damage in the myocardial cells were graded according to the method according to Billingham et al. (Can. Treat. Rep. 62: 865-872, 1978). In brief, blocks were selected that consisted predominantly of transected cardiac myocytes. Lesions were scored, and the extent of cardiac damage was expressed as a percentage of vacuolated myocytes.

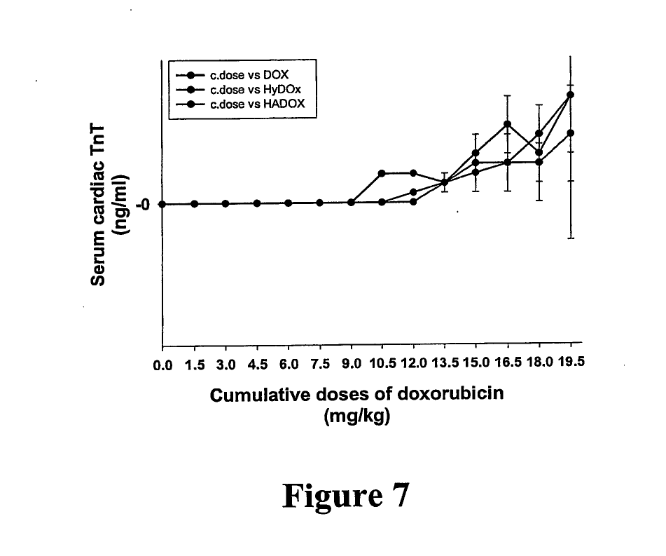

FIG. 7 shows the effect of HA/Dox on cardiotoxicity. As indicated by this graph, the formulation of HA with Dox or injection of HA before drug can delay the onset of cardiotoxicity. The serum troponin T data from the rat study, at this stage appears that HyDox does have an effect of delaying the release of Troponin T (the cumulative dose of Dox is 9 mg/kg while cardiotoxicity of HyDox begins at 12 mg/kg cumulative dose.)

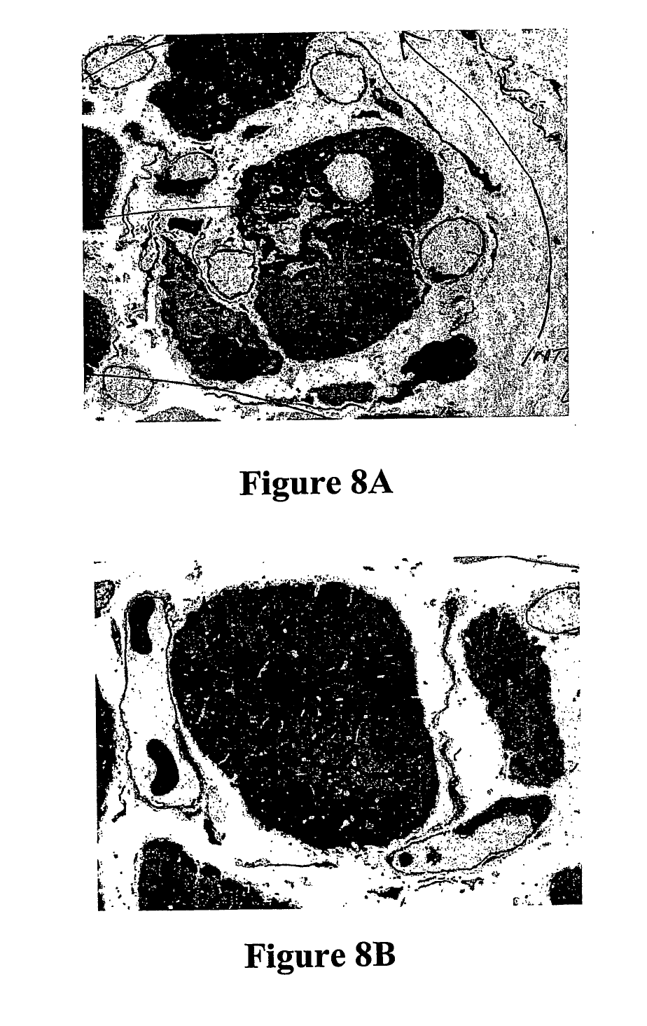

FIGS. 8A-8B shows the effect of HA and Dox on Cardiotoxicity in rats. It should be noted that the vacuolated myocytes in the hearts of animals which received Dox versus the non-vacuolated healthy myocytes of animals treated with HA and Dox.

### EXAMPLE 3

### In vivo Model of Cytotoxicity (II)

Experimental Animals

Male F1 mice (C57×CBA, 11-13 weeks old) were obtained from The Animal Central Division (Monash University, Victoria, Australia). For each treatment dosage a total of 6 groups (n=8 mice per group) were used. Data from each treatment group were established by staggering results of groups of 6 (counted individually), sampling each group every 4 days so that a daily assessment could be established. Each group was treated as described above, prior to the drug administration. Throughout this study, blood was collected and analysed for neutrophil content as outlined in previous examples. At the end point, all animals were humanely sacrificed and body organs fixed in 10% v/v formalin in PBS.

Hematological Determinations

This study focused on the two dosages of 12 mg/kg Dox and 16 mg/kg Dox that are the human therapeutic equivalents of 60 and 80 mg/m², respectively. Again, to normalize the data for comparison between treatments, neutropenia was expressed as a percentage of neutrophils on day 0 (before drug administration).

12 mg/kg Doxorubicin and HyDox Treatment Groups

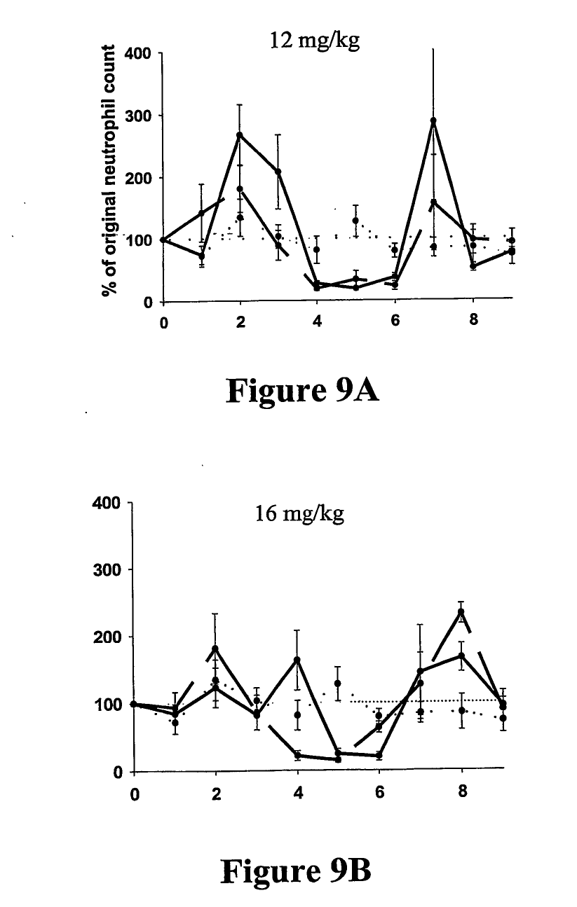

At a dosage of 12 mg/kg, a trend similar to neutropenia in Example 1 was observed (FIG. 9, panel 12 mg/kg). Despite a drop in neutrophil content 24 hours after HyDox administration to approximately 74%, by days 2 and 3 there was an average increase in peripheral neutrophils to 267% and 207%, respectively. Although an increase in peripheral neutrophils, which peeked at day 2, was observed in the Dox only group, it never reached the maximum value obtained in the HyDox treated groups. The onset and duration of nadir neutropenia was equally severe in both groups: it occurred initially at day 4 and lasted for 2 days before recovering to normal levels by day 7.

16 mg/kg Doxorubicin and HyDox Treatment Groups

At the Dox dosage of 16 mg/kg, a similar neutropenia profile was observed irrespective of drug combination tested (FIG. 9). Despite this trend, the onset of neutropenia was delayed by one day in those groups treated with the HyDox formulation (FIG. 9, panel 16 mg/kg, solid line) and occurred at day 5. Nadir neutropenia was equal in both combinations of drug administration but only lasted for a period of 24 hours in those groups treated with HyDox, before recovery to normal measurement. In contrast, in mice treated with Dox, the recovery from nadir neutropenia took longer than 48 hours.

This was followed by a decline in peripheral neutrophils and by day 4 nadir neutropenia was comparable between drug combinations, lasted for 48 hours before returning to normal measurements.

In mice treated with 16 mg/kg Dox, in either drug combination, the same neutropenia profile was observed. Both groups displayed an increase in peripheral neutrophils by day 2 following iv administration of drug, which preceded a decline in neutrophil numbers. In mice receiving 16 mg/kg HyDox, however, this decline was delayed by 24 hours but was equally severe when compared with mice receiving Dox only (FIG. 9).

Other Haematological Considerations

Analysis of the differential results for red blood cell count, haemoglobin, platelets and lymphocytes did not show any difference in effect when Dox was administered as either HyDox or drug alone.

Metabolic Stress

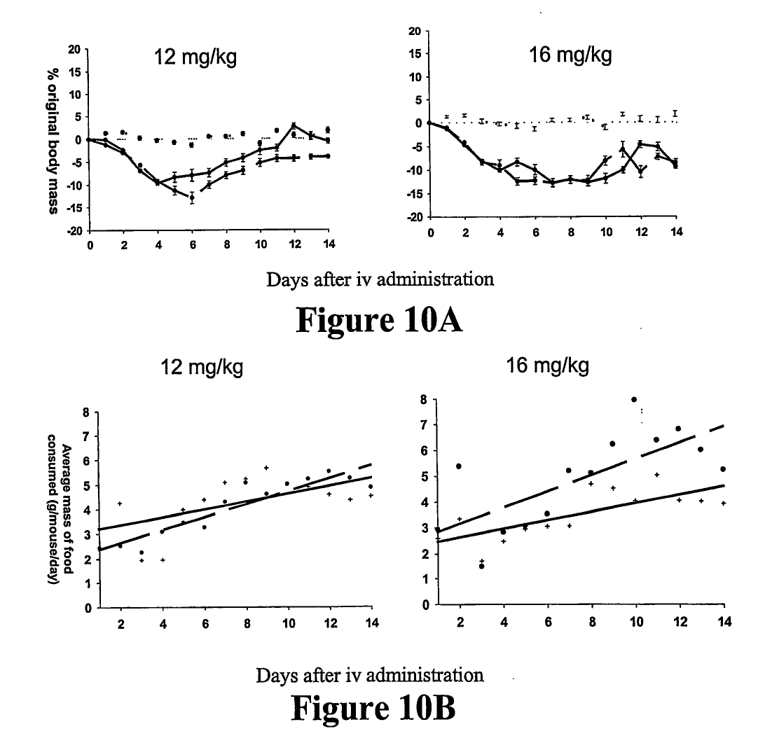

Weight-loss was expressed as a percentage of the original starting mass. Weight loss was equal up to day 4, in groups treated with 12 mg/kg Dox or HyDox. By day 5, however, the HyDox group started to re-gain weight and by day 12 had recovered to original starting mass (FIG. 10A, 12 mg/kg). In contrast, animals treated with 12 mg/kg Dox continued to lose weight until day 6, before starting to re-gain weight. In the 12 mg/kg dosage groups, the maximum percentage of weight lost was 12.9% and occurred at day 6, whereas the HyDox group only lost a maximum of 9.6% by day 4. Mice receiving drug only never regained their original starting FIG. 10A, 12 mg/kg).

The weight loss in mice treated with 16 mg/kg Dox and HyDox formulation followed a similar trend, and both groups never re-gained their original body mass. It is interesting note, however that despite equal weight loss between the two groups, mice receiving drug only consumed more food on average throughout the duration of the study (FIG. 10B, 16 mg/kg Dox). This effect was even more noticeable in the groups receiving HyDox at the drug dosage of 12 mg/kg. The HyDox group regained weight faster when compared with the drug alone data, yet consumed equal amounts of food throughout the study (FIG. 10B, 12 mg/kg).

### EXAMPLE 4

### In vivo Model of Cardiotoxicity (II)

Drugs for Intravenous Injection and Other Chemicals

Doxorubicin was purchased from Asta Medical (NSW, Australia) as doxorubicin hydrochloride powder, which was reconstituted in 0.9% sterile sodium chloride to a final concentration of 2 mg/mL. Desiccated HA, 824,000 Da, was purchased from Pearce Pharmaceuticals (Victoria, Australia) and was dissolved in sterile water to a final concentration of 10 mg/mL, filter sterilized through a 0.22 μM filter and stored at 4° C. until used. HyDox was prepared by mixing calculated volumes of 10 mg/mL HA with a calculated volume of 0.5 mg/mL Dox to achieve the desired 1.0 mg/kg Dox dosage.

The dosage of HA used throughout this study was 13.3 mg/kg of bodyweight. Glutathione, o-phthalaldehyde, xanthine, xanthine oxidase (from Buttermilk, Grade I), reduced NADPH type III were purchased from Sigma Chemicals (St. Louis, Mo.). Catalase, glutathione peroxidase and glutathione reductase were purchased from Roche Molecular Biochemicals ASW, Australia). All other reagents were of analytical grade.

Experimental Animals

Adult female spontaneously hypertensive rats (10 weeks old) were purchased from The Baker Medical Research Institute (Melbourne, Australia) and were randomly divided into 4 experimental groups (n=10 per treatment group). Groups 1 and 2 received weekly intravenous injections of: (1) 1.0 mg/kg Dox only: (2) 1.0 mg/kg HyDox. Group 3 received weekly intravenous injections of 13.3 mg/kg HA. Group 4 received no treatment. Animals were injected on weekly intervals with drug combinations for 12 weeks. The total cumulative dose of Dox received in both treatment groups was 13 mg/kg.

For analyses, tissue processing and assays of antioxidant enzymes and GSH, were carried out as described in Example 2. Analysis of cardiac damage by transmission electron microscopy was also carried out as described in Example 2.

Myocardial Pathology, Ultra-Structural Studies

To develop a model for induced chronic cardiotoxicity, weekly iv injections of 1.0 mg/kg Dox, as either drug alone or HyDox, were administered to rats. The total cumulative dose of Dox given to experimental groups was 13 mg/kg. Electron microscopy was then used to assess whether chronic exposure to Dox, with or without the presence of HA, was cardioprotective or cardiotoxic, respectively. To minimize anatomical variation across treatment groups, samples of the lateral border of the left ventricle were processed for EM analysis.

Electron microscopy examination revealed that a cumulative dose of 13 mg/kg Dox was cardiotoxic in the spontaneously hypertensive rat model and was consistent with other studies of similar subject. Specifically, Dox-cardiotoxicity presented primarily as cytoplasmic vacuolation, myofibrillar disorganization and disruption to the ultrastructure organization of myocytes.

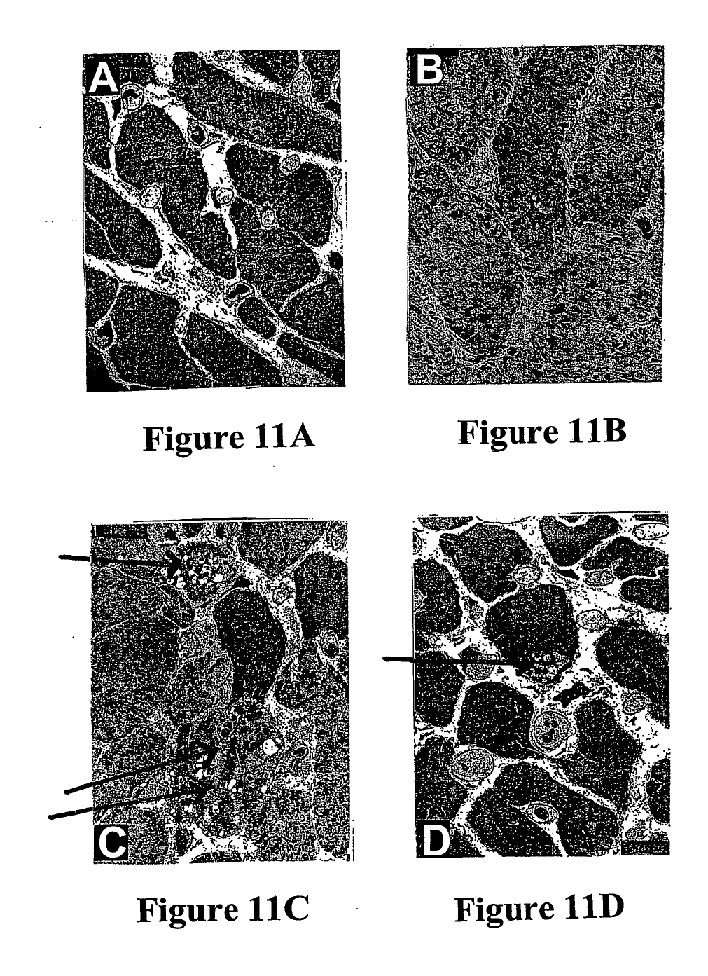

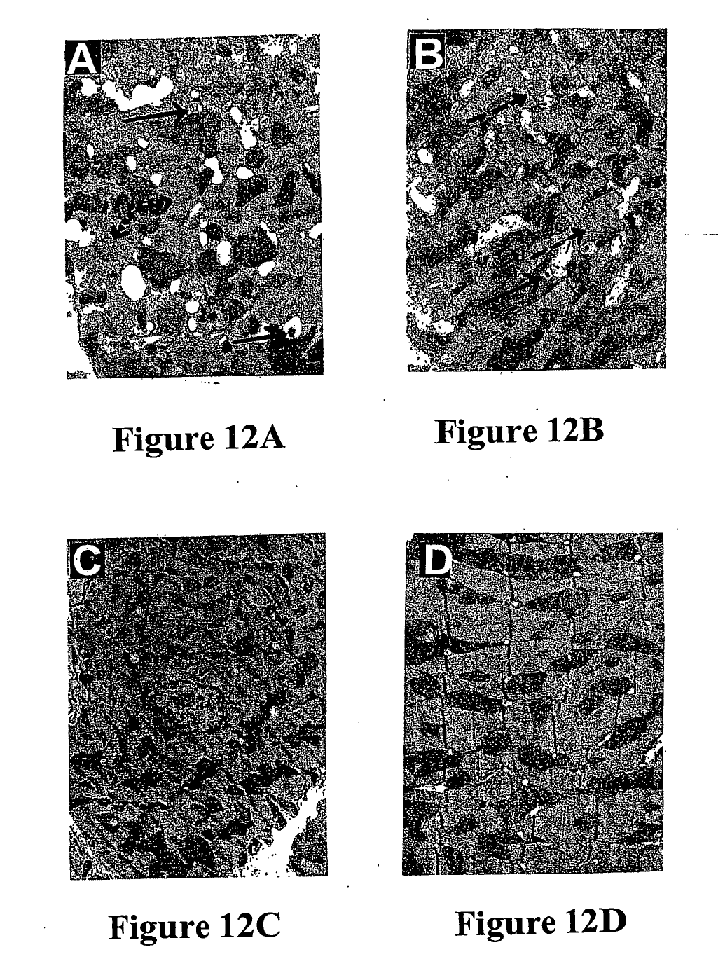

In rats treated with Dox, the occurrence of cardiomyocyte vacuolation was far more frequent when compared with micrographs obtained from rats receiving HyDox (FIGS. 11C and 11D, respectively). Dox also caused mitochondrial swelling with minor disruption to the cristae. However, the severity was greater in rats receiving Dox only. In fact, the degenerative mitochondrial changes were associated with complete disruption to these sub-cellular organelles and were consistent with the appearance of myelin figures (FIGS. 12A and 12B, solid arrows). These observations were less frequent in rats receiving HyDox (FIG. 12B). Chronic exposure to Dox also caused mild disruption to the organized myofibrillar array that included myofibrillar lysis and misalignment of the Z-bands. Again these features of Dox cardiotoxicity were always more severe in rats receiving Dox only (FIG. 12A, dotted arrows). Although myofibrillar lysis was still evident, it was less severe in rats treated with HyDox and ordered myofibril arrays were still evident (FIG. 12B, dashed arrows).

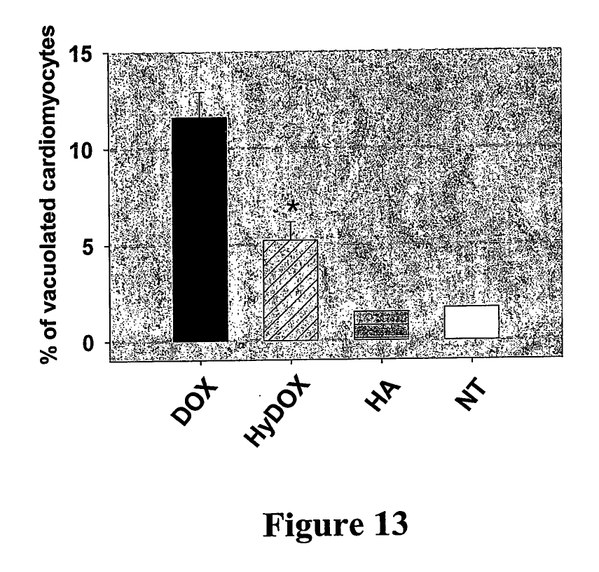

The severity of cardiomyocyte vacuolation in all treatment groups is shown in FIG. 13. When compared with the HyDox group, myocyte vacuolation in the Dox group is almost two-three-fold greater (p<0.05: t-test between treatment groups).

Antioxidant Levels Following Chronic Exposure to Dox±HA

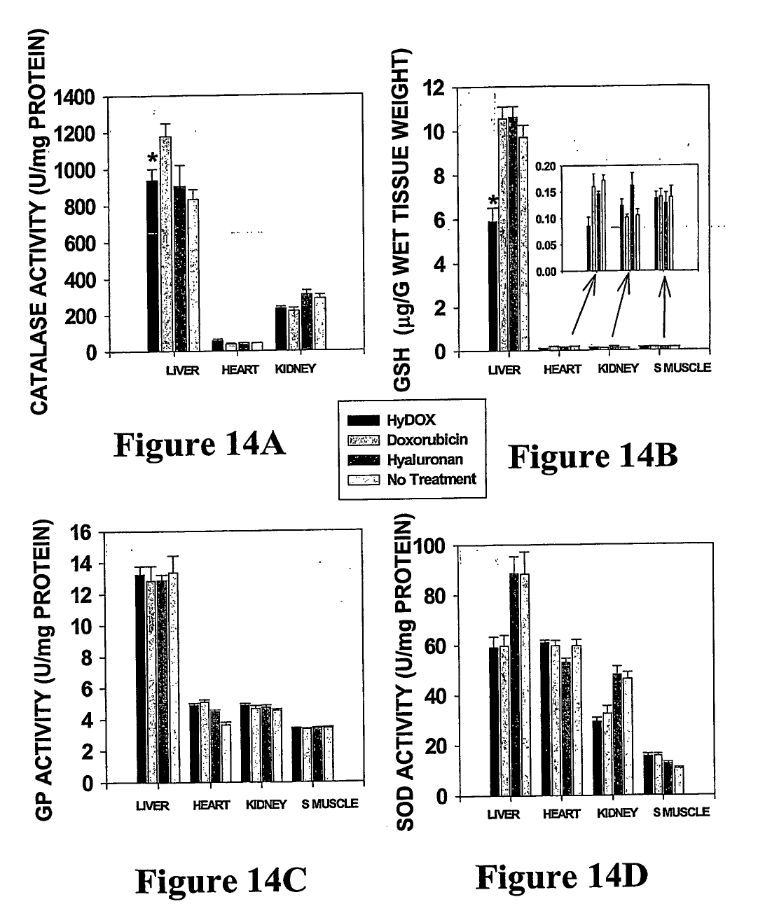

The levels of free-radical scavenging enzymes; namely, superoxide dismutase, catalase, glutathione peroxidase, and levels of reduced glutathione (GSH), were measured after chronic exposure to Dox±HA. For control purposes, antioxidant loading from hepatic, renal and skeletal muscle extracts was also determined. The effect of chronic exposure to Dox and HyDox on the cardiac, hepatic, renal and skeletal antioxidant capacity is shown in FIG. 14.

Catalase

A slight increase in cardiac catalase in response to HyDox was observed when compared with Dox and both control groups. Hepatic catalase was significantly increased in response to Dox yet the HyDox activity mirrored those observed from both control groups (FIG. 14A).

Reduced Glutathione (GSH)

The level of hepatic GSH was significantly reduced in rats receiving HyDox. However, the GSH levels in Dox-treated rats remained unchanged in relation to the control values (FIG. 14B). A similar trend was observed in cardiac levels where HyDox reduced the GSH content yet Dox treatment yielded similar values to the controls (FIG. 14B: insert with hepatic data removed).

Glutathione Peroxidase

Both HyDox and Dox treatment equally increased the level of cardiac glutathione peroxidase levels (FIG. 14C) when compared with the values obtained from the control groups. Both drug-combinations resulted in similar hepatic, renal and skeletal glutathione peroxidase activities when compared with corresponding control data (FIG. 14C).

Superoxide Dismutase (SOD)

Cardiac SOD activity remained relatively unchanged between treatment groups when compared with the no treatment control group (FIG. 14D). The HA control group significantly decreased the activity of this enzyme (FIG. 14D). Suppression of hepatic and renal SOD was observed in both treatment groups, whereas skeletal SOD activity was moderately increased (FIG. 14D). Hepatic catalase activity was significantly increased in response to Dox exposure, yet the HyDox activity mirrored those observed from both the control groups (FIG. 14A).

Cardiotoxicity is a complicating factor that limits the total cumulative dose of Dox chemotherapy to 500-550 mg/m². These data demonstrate that, by combining Dox with HA (HyDox), the level of cardiotoxicity is reduced almost three-fold when compared with drug alone. Hence, the use of HyDox enables a higher cumulative dose of the highly efficacious anti-cancer drug, without the normally encountered cardiotoxicity. Completion of treatment without cardiotoxicity enhances the probability of tumor response and ultimately increases survival.

### EXAMPLE 5

### Phase I Clinical Trials—Combination of Doxorubicin+HA (“HyDox”) in Patients with Advanced Cancer

A phase I study was conducted to determine whether co-administration of HA with Dox impacted on Dox toxicity in patients with advanced cancer.

The eligibility criteria, for inclusion in this study, were as follows: Patients must have advanced or metastatic cancer, histologically or cytologically confirmed. Patients were not to have had previous chemotherapy or were to have had no more than one prior chemotherapy regime and were not to have had prior anthracyclines. Patients were to be aged between 18 and 75 years with ambulatory performance status and adequate bone marrow, liver and renal function.

The treatment plan provided that, in the first chemotherapy cycle, patients be randomized to receive Dox alone or Dox plus HA. The converse applied in the second cycle. In the subsequent four cycles, patients received Dox plus HA. This allowed each patient to act as his/her own control in the toxicity analysis in the first two cycles, reducing the possibility of inter-patient variability affecting result interpretation.

The HyDox formulation (as infusion bags) was prepared as follows: HA was obtained from GlycoMed Research, New York, USA and used from a 10 mg/ml stock prepared from dissolving powdered HA in distilled water and filter sterilising through a 0.22 μm filter. Dox was obtained from Asta Medica, supplied as a 50 mg vial containing 250 mg lactose, and was reconstituted in 25 ml of injectable normal saline, by constant swirling for 8 to 12 minutes. Injectable sodium chloride was obtained from Baxter Healthcare, Sydney, Australia, as supplied as a 500 ml infusion bag. The injectable HyDox was prepared to deliver 13.3 mg/kg HA with 30, 45 or 60 mg/m² Dox.

The Dox±HA was given by intravenous administration over one hour on a three week cycle. The initial dose of Dox in this phase 1 study was 30 mg/m² and the dose of HA 500 mg/m². Dose escalation occurred in two steps to reach the standard dose of 60 mg/m². Therefore, the initial dose level was 30 mg/m², the next 45 mg/m² and the next 60 mg/m².

Four patients, at each dose level were treated. The results demonstrated that HA does not increase any of the known toxicities of Dox. At the higher dose level, 60 mg/m², was a lessening of nausea and vomiting post-chemotherapy was apparent, as well as a reduction of both hair loss and the extent of neutropenia induced by Dox. The results of neutrophil. counts are shown in Table 9. The patient's well-being appeared to be improved. No other toxicities were reported.

TABLE 9
Patient 1: A-S#1

| Cycle 1: | 30 mg/m2 | | Cycle 2: | 30 mg/m2 | | Cycle 3: | 30 mg/m2 | |
|---|---|---|---|---|---|---|---|---|
| HyDOX | Neutrophils X | Neutrophils as | DOX | Neutrophils X | Neutrophils as | DOX | Neutrophils X | Neutrophils as |
| Time (days) | 109/L | % of Day 1 | Time (days) | 109/L | % of Day 1 | Time (days) | 109/L | % of Day 1 |
| 1 | 3.6 | 100 | 1 | 3.5 | 100 | 1 | 2.6 | 100 |
| 3 | 4.5 | 125 | 6 | 3.5 | 100 | | | 0 |
| 6 | 5.7 | 158 | 8 | 3.8 | 109 | | | 0 |
| 8 | 5 | 139 | 10 | 3.8 | 109 | | | 0 |
| 10 | 5.1 | 142 | 13 | 2.9 | 83 | | | 0 |
| 15 | 2.9 | 81 | 15 | 2 | 57 | | | 0 |
| 17 | 2.3 | 64 | 20 | 2 | 57 | | | 0 |
| 20 | 2.7 | 75 | 21 | 2.6 | 74 | | | |
| 21 | 3.5 | 97 | | | | | | |

Patient 2: H-Y#2

| Cycle 1: | 30 mg/m2 | | Cycle 2: | 30 mg/m2 | | Cycle 3: | 30 mg/m2 | |
|---|---|---|---|---|---|---|---|---|
| DOX | Neutrophils X | Neutrophils as | HyDOX | Neutrophils X | Neutrophils as | DOX | Neutrophils X | Neutrophils as |
| Time (days) | 109/L | % of Day 1 | Time (days) | 109/L | % of Day 1 | Time (days) | 109/L | % of Day 1 |
| 1 | 5 | 100 | 1 | 4.3 | 100 | 1 | 3 | 100 |
| 4 | 6 | 120 | 4 | 5.3 | 123 | | | 0 |
| 8 | 6.6 | 132 | 8 | 6.4 | 149 | | | 0 |
| 10 | 3.9 | 78 | 11 | 4 | 93 | | | 0 |
| 15 | 2.7 | 54 | 12 | 4.2 | 98 | | | 0 |
| 17 | 2 | 40 | 15 | 4.1 | 95 | | | 0 |
| 21 | 4.3 | 86 | 17 | 2.3 | 53 | | | 0 |
| | | | 21 | 3 | 70 | | | |

Patient 5: A-L#5

| Cycle 1: | 30 mg/m2 | | Cycle 2: | 30 mg/m2 | | Cycle 3: | 30 mg/m2 | |
|---|---|---|---|---|---|---|---|---|
| DOX | Neutrophils X | Neutrophils as | HyDOX | Neutrophils X | Neutrophils as | DOX | Neutrophils X | Neutrophils as |
| Time (days) | 109/L | % of Day 1 | Time (days) | 109/L | % of Day 1 | Time (days) | 109/L | % of Day 1 |
| 1 | 3.7 | 100 | 1 | 2.2 | 100 | 1 | 2.1 | 100 |
| 4 | 2.9 | 78 | 4 | 3.1 | 141 | | | |
| 7 | 2.6 | 70 | 7 | 3.2 | 145 | | | |
| 9 | 2.7 | 73 | 9 | 3.5 | 159 | | | |
| 12 | 1.8 | 49 | 12 | 1.8 | 82 | | | |
| 14 | 1.4 | 38 | 14 | 1.6 | 73 | | | |
| 16 | 1.2 | 32 | 16 | 1.4 | 64 | | | |
| 19 | 1.4 | 38 | 19 | 1 | 45 | | | |
| 21 | 2.2 | 59 | 21 | 1.9 | 86 | | | |

Patient 7: W-H#7

| Cycle 1: | 45 mg/m2 | | Cycle 2: | 45 mg/m2 | | Cycle 3: | 45 mg/m2 | |
|---|---|---|---|---|---|---|---|---|
| DOX | Neutrophils X | Neutrophils as | HyDOX | Neutrophils X | Neutrophils as | DOX | Neutrophils X | Neutrophils as |
| Time (days) | 109/L | % of Day 1 | Time (days) | 109/L | % of Day 1 | Time (days) | 109/L | % of Day 1 |
| 1 | 6.3 | 100 | 1 | 3.2 | 100 | 1 | 2.6 | 100 |
| 4 | 2.9 | 46 | 4 | 2.8 | 88 | | | |
| 8 | 3.5 | 56 | 10 | 2.5 | 78 | | | |
| 10 | 2.6 | 41 | 12 | 1.6 | 50 | | | |
| 12 | 2.2 | 35 | 17 | 1.3 | 41 | | | |
| 15 | 1.2 | 19 | 19 | 1.5 | 47 | | | |
| 17 | 0.9 | 14 | | | | | | |
| 19 | 1.3 | 21 | | | | | | |

Patient 8: S-W#8

| Cycle 1: | 45 mg/m2 | | Cycle 2: | 45 mg/m2 | | | 45 mg/m2 | |
|---|---|---|---|---|---|---|---|---|
| HyDOX | Neutrophils X | Neutrophils as | DOX | Neutrophils X | Neutrophils as | Cycle 3: | Neutrophils X | Neutrophils as |
| Time (days) | 109/L | % of Day 1 | Time (days) | 109/L | % of Day 1 | Time (days) | 109/L | % of Day 1 |
| 1 | 4 | 100 | 1 | 5.7 | 100 | 1 | | #DIV/0! |
| 4 | 7.3 | 183 | 4 | 4.8 | 84 | | | |
| 8 | 4.9 | 123 | 8 | 4.1 | 85 | | | |
| 10 | 2.2 | 55 | 10 | 1.6 | 39 | | | |
| 12 | 1.1 | 28 | 12 | 1.3 | 81 | | | |
| 17 | 1.9 | 48 | 15 | 0.8 | 62 | | | |
| 19 | 3.1 | 78 | | | 0 | | | |
| | | | | | #DIV/0! | | | |

Patient 9: J-V#9

| Cycle 1: | 60 mg/m2 | | | 60 mg/m2 | | | 60 mg/m2 | |
|---|---|---|---|---|---|---|---|---|
| HyDOX | Neutrophils X | Neutrophils as | Cycle 2: | Neutrophils X | Neutrophils as | Cycle 3: | Neutrophils X | Neutrophils as |
| Time (days) | 109/L | % of Day 1 | Time (days) | 109/L | % of Day 1 | Time (days) | 109/L | % of Day 1 |
| 1 | 4.9 | 100 | 1 | 9.9 | 100 | 1 | | #DIV/0! |
| 4 | 6.2 | 127 | 4 | 13.8 | 139 | | | |
| 8 | 5.6 | 114 | 7 | 14.7 | 107 | | | |
| 10 | 2.2 | 45 | | | | | | |
| 12 | 0.7 | 14 | | | | | | |
| 15 | 0.8 | 16 | | | | | | |
| 17 | 2.4 | 49 | | | | | | |
| 19 | 5.5 | 112 | | | | | | |

Patient 10: E-C#10

| Cycle 1: | 60 mg/m2 | | Cycle 2: | 60 mg/m2 | | | 60 mg/m2 | |
|---|---|---|---|---|---|---|---|---|
| DOX | Neutrophils X | Neutrophils as | HyDOX | Neutrophils X | Neutrophils as | Cycle 3: | Neutrophils X | Neutrophils as |
| Time (days) | 109/L | % of Day 1 | Time (days) | 109/L | % of Day 1 | Time (days) | 109/L | % of Day 1 |
| 1 | 7.6 | 100 | 1 | 4.5 | 100 | 1 | | #DIV/0! |
| 4 | 7.1 | 93 | 4 | 4 | 89 | | | |
| 6 | 4.8 | 63 | 6 | 3.3 | 73 | | | |
| 8 | 1.6 | 33 | | | | | | |
| 10 | 0.3 | 4 | | | | | | |
| 13 | 0.2 | 3 | | | | | | |
| 17 | 1.5 | 20 | | | | | | |
| 20 | 3.6 | 47 | | | | | | |

Patient 11: H-T#11

| Cycle 1: | 60 mg/m2 | | | 60 mg/m2 | | | 60 mg/m2 | |
|---|---|---|---|---|---|---|---|---|
| HyDOX | Neutrophils X | Neutrophils as | Cycle 2: | Neutrophils X | Neutrophils as | Cycle 3: | Neutrophils X | Neutrophils as |
| Time (days) | 109/L | % of Day 1 | Time (days) | 109/L | % of Day 1 | Time (days) | 109/L | % of Day 1 |
| 1 | 3 | 100 | 1 | 2.4 | 100 | 1 | | #DIV/0! |
| 4 | 2.1 | 70 | 4 | | | | | |
| 8 | 2 | 67 | 7 | | | | | |
| 10 | 1.5 | 50 | | | | | | |
| 12 | 0.8 | 27 | | | | | | |
| 15 | 0.5 | 17 | | | | | | |
| 17 | 0.5 | 17 | | | | | | |
| 19 | 0.9 | 30 | | | | | | |

Patient 12: M-P#12

| Cycle 1: | 60 mg/m2 | | | 60 mg/m2 | | | 60 mg/m2 | |
|---|---|---|---|---|---|---|---|---|
| DOX | Neutrophils X | Neutrophils as | Cycle 2: | Neutrophils X | Neutrophils as | Cycle 3: | Neutrophils X | Neutrophils as |
| Time (days) | 109/L | % of Day 1 | Time (days) | 109/L | % of Day 1 | Time (days) | 109/L | % of Day 1 |
| 1 | 5.8 | 100 | | | | 1 | | #DIV/0! |
| 4 | 7.2 | 124 | | | | | | |
| 7 | 5.5 | 95 | | | | | | |

### EXAMPLE 6

### Phase I Clinical Trials—Combination of 5-Fluorouracil+HA (“Hyfive”) in Patients with Metastatic Colorectal Cancer

A similar phase I study was carried out with a combination of HA and 5-fluorouracil (Hyfive) in patients with metastaic colorectal cancer. The eligibility criteria were as for Example 3, except that anthracyclines were not contraindicated. The treatment plan proceeded as set out for Example 3.

The HyDox formulation (as infusion bags) was prepared as follows: HA and injectable sodium chloride were obtained from GlycoMed Research and Baxter Healthcare respectively, as set out in Example 3. Dox was obtained from David Bull Laboratories, and supplied as a 10 ml vial containing 500 mg 5-fluorouracil (5-FU).

The 5-FU±HA was given by intravenous administration over one hour on a four week cycle. The initial dose of 5-FU in this study was 450 mg/m² daily for three days and the dose of HA 500 mg/m² with each administration of 5-FU. Dose escalation occurred in two steps to reach the standard dose of 450 mg/m² for five consecutive days with each cycle. Therefore, the initial dose level was 450 mg/m² daily for three days, the next 450 mg/m² for four days and the final 450 mg/m² daily for five days.

Four patients, at each dose level, were treated. The results demonstrated that HA does not increase any of the known toxicities of 5-FU. At the higher dose level there was an apparent lessening of nausea and vomiting post-chemotherapy, and there was no neutropenia or gastrointestinal tract toxicities. The results of neutrophil counts are shown in Table 10.

TABLE 10
Patient 2 - L-F (Day 3 Tx)

| Cycle 1 - 5FU | Neutrophil | | Cycle 2 - HyFive | | | Cycle 3 - HyFive | | |
|---|---|---|---|---|---|---|---|---|
| Time (Days) | Count | as % Day 1 | Time (Days) | Neutrophil Count | as % Day 1 | Time (Days) | Neutrophil Count | as % Day 1 |
| 1 | 9.3 | 100 | 1 | 8.7 | 100 | 2 | 9.6 | |
| 3 | 7.6 | 82 | 3 | 8.1 | 93 | | | |
| 7 | 8.4 | 90 | 7 | 8.1 | 93 | | | |
| 9 | 8.3 | 89 | 9 | 7.5 | 86 | | | |
| 11 | 6.8 | 73 | 11 | 7.2 | 83 | | | |
| 14 | 7 | 75 | 14 | 7.7 | 89 | | | |
| 16 | 7.3 | 78 | 16 | 10.8 | 124 | | | |
| 18 | 8.4 | 90 | 18 | 7.9 | 91 | | | |
| 21 | 8.2 | 88 | 21 | 7.9 | 91 | | | |
| 23 | 7.8 | 84 | 23 | 7.9 | 91 | | | |
| 25 | 8.7 | 94 | 25 | 8.4 | 97 | | | |

Patient 4 - C-X (4 Day Tx)

| Cycle 1 - | | | | | | | | |
|---|---|---|---|---|---|---|---|---|
| HyFive | Neutrophil | | Cycle 2 - 5FU | | | Cycle 3 - HyFive | | |
| Time (Days) | Count | as % Day 1 | Time (Days) | Neutrophil Count | as % Day 1 | Time (Days) | Neutrophil Count | as % Day 1 |
| 1 | 5.8 | 100 | 1 | 5.1 | 100 | 1 | 5.5 | |
| 4 | 6.1 | 105 | 4 | 4.4 | 86 | | | |
| 8 | 10.2 | 176 | 5 | 4.4 | 86 | | | |
| 10 | 10.8 | 186 | 8 | 5 | 98 | | | |
| 15 | 14.3 | 247 | 10 | 10.3 | 202 | | | |
| 17 | 13.1 | 226 | 12 | 6.9 | 135 | | | |
| 19 | 12.2 | 210 | 15 | 6.7 | 131 | | | |
| 22 | 6 | 103 | 17 | 7.5 | 147 | | | |
| 24 | 5.8 | 100 | 19 | 6.5 | 127 | | | |
| | | | 22 | 6.1 | 120 | | | |
| | | | 24 | 7.9 | 155 | | | |
| | | | 26 | 5.3 | 104 | | | |

Patient 5 - J-M (5 Day Tx)

| Cycle 1 - 5FU | Neutrophil | | Cycle 2 - HyFive | | | Cycle 3 - HyFive | | |
|---|---|---|---|---|---|---|---|---|
| Time (Days) | Count | as % Day 1 | Time (Days) | Neutrophil Count | as % Day 1 | Time (Days) | Neutrophil Count | as % Day 1 |
| 1 | 4.2 | 100 | 1 | 4 | 100 | 1 | 5.8 | |
| 5 | 4.5 | 107 | 3 | 3.9 | 98 | | | |
| 10 | 4.1 | 98 | 7 | 4.8 | 120 | | | |
| 12 | 4.3 | 102 | 9 | 5.6 | 140 | | | |
| 15 | 3.7 | 88 | 11 | 7.1 | 178 | | | |
| 17 | 3.3 | 79 | 14 | 5.8 | 145 | | | |
| 19 | 4.3 | 102 | 16 | 5.6 | 140 | | | |
| 22 | 4.3 | 102 | 18 | 4.5 | 113 | | | |
| 24 | 4 | 95 | 21 | 5.2 | 130 | | | |
| 26 | 4.5 | 107 | 23 | 5.5 | 138 | | | |
| | | | 25 | 5.7 | 143 | | | |

Patient 6 - J-B (5 Day Tx)

| Cycle 1 - 5FU | Neutrophil | | Cycle 2 - HyFive | | | Cycle 3 - | | |
|---|---|---|---|---|---|---|---|---|
| Time (Days) | Count | as % Day 1 | Time (Days) | Neutrophil Count | as % Day 1 | Time (Days) | Neutrophil Count | as % Day 1 |
| 1 | 5.3 | 100 | 1 | 4.5 | 100 | 1 | 6.1 | |
| 3 | 4.3 | 81 | 3 | 4.9 | 109 | | | |
| 8 | 4.5 | 85 | 5 | 4.6 | 102 | | | |
| 10 | 6.6 | 125 | 8 | 7.5 | 167 | | | |
| 12 | 4.5 | 85 | 10 | 8.6 | 191 | | | |
| 15 | 4.2 | 79 | 12 | 5.8 | 129 | | | |
| 17 | 5.5 | 104 | 13 | 7 | 156 | | | |
| 19 | 5.3 | 100 | 19 | 6 | 133 | | | |
| 22 | 5.7 | 108 | 22 | 7 | 156 | | | |
| 24 | 5.4 | 102 | 24 | 5.5 | 122 | | | |
| 26 | 5 | 94 | 26 | 5.5 | 122 | | | |

Patient 7 - V-D (5 Day Tx)

| Cycle 1 - | | | | | | | | |
|---|---|---|---|---|---|---|---|---|
| HyFive | Neutrophil | | Cycle 2 - 5FU | | | Cycle 3 - | | |
| Time (Days) | Count | as % Day 1 | Time (Days) | Neutrophil Count | as % Day 1 | Time (Days) | Neutrophil Count | as % Day 1 |
| 1 | 2.7 | 100 | 1 | 3 | 100 | 1 | 3 | |
| 3 | 3.2 | 119 | 3 | 2.3 | 77 | | | |
| 5 | 2.7 | 100 | 5 | 2.8 | 93 | | | |
| 8 | 3.3 | 122 | 8 | 3.2 | 107 | | | |
| 10 | 2.5 | 93 | 10 | 2.5 | 83 | | | |
| 12 | 2.6 | 96 | 15 | 2.7 | 90 | | | |
| 19 | 2.5 | 93 | 17 | 2.5 | 83 | | | |
| 22 | 2.4 | 89 | 19 | 3 | 100 | | | |
| 24 | 2.9 | 107 | 22 | 3.4 | 113 | | | |
| 26 | 2.9 | 107 | 24 | 3.1 | 103 | | | |
| | | | 26 | 3.5 | 117 | | | |

### EXAMPLE 7

### In vivo Model of Cytotoxicity (III)

Examples 5 and 6 investigated the effect of HA/cytotoxic drug combinations for a 6-month period and a 6-week period, respectively, and showed that the addition of HA to methotrexate or 5-FU enhanced tumor response, reduced metastasis and reduced gastrointestinal toxicity. Those results were followed up with an investigation of the effect of HA on the efficacy of cyclophosphamide, methotrexate and 5-flurouracil (CMF) in the treatment of human breast cancer xenografts in nude mice. The following efficacy parameters were investigated: primary tumor volume; cancer metastasis and treatment toxicity in relation to body mass, organ pathology, hematology and survival.

Humant Breast Carcinoma Cell Line

Human breast carcinoma cell line MDA-MB-468 (American Tissue Culture Collection, Rockville, USA) was selected based on its expression of the HA receptors of CD44, and RHAMM. Cells were routinely grown and subcultured as a monolayer in 175 cm² culture flasks or 700 cm² roller bottles in Leibovitz L-15 Medium (Sigma. St. Louis, USA) supplemented with 10% w/v fetal calf serum and 10 μg/ml gentamycin. For injection into mice, cells were grown to 80% confluency, trypsinized in 0.05% trypsin/0.01% EDTA solution, washed twice by centrifugation in a Beckman TJ-6 bench centrifuge (Beckmnan, Melbourne, Australia) at 400 gav for 10 minutes, counted using a Model-ZM Coulter counter (Coulter Electronics, England) and resuspended in serum-free Leibovitz L-15 medium at 2×10⁸ cell/ml.

Mouse Tumor Model

Athymic CBA/WEHI nude female mice (Walter and Eliza Hall Research Institute, Melbourne, Australia), 6 to 8 weeks old, were maintained under specific pathogen-free conditions, with sterilized food and water available ad libitum. Each mouse received one injection containing 1×10⁷ cells in 50-100 μl. The cells were injected with a 26-gauge needle into the mammary fat pad directly under the first nipple (Lamszus et al., Lab. Invest. 76(3): 339-353, 1997). Tumor measurements were made weekly by measuring three perpendicular diameters (d₁d₂d₃). Tumor volume was estimated using the formula:
(⅙)π(d₁d₂d₃).

Treatment with CMF±HA was commenced approximately 4-8 weeks after the cancer cell inoculation. Table 11 shows the mean tumor volume of each treatment group at commencement of treatment.

TABLE 11

| | Tumor volume (mm³) | Tumor volume (mm³) as |
|---|---|---|
| | at commencement | percentage of body mass |
| | of treatment | at commencement of |
| Treatment Group | Mean ± SD | treatment Mean ± SD |
| Saline Day 1 & 2 | 39.98 ± 13.88 | 0.20 ± 0.07 |
| HA Day 1 & 2 | 50.99 ± 18.07 | 0.22 ± 0.10 |
| HA Day 1 & 3 | 28.65 ± 20.90 | 0.14 ± 0.11 |
| CMF Day 1 & 2 | 44.51 ± 26.92 | 0.23 ± 0.13 |
| HA/CMF Day 1 & 2 | 37.13 ± 22.30 | 0.20 ± 0.12 |
| HA days 1 & 3 | 35.31 ± 9.42 | 0.20 ± 0.05 |
| followed by | | |
| CMF days 2 & 4 | | |

Animal Maintenance and Housing

Equal numbers of mice were allocated to each cage. The animal number per cage varied from 5-8, depending on the stage of experimentation. All animals were housed at the Monash University Central Animal Services SPF facility in accordance with internal quality assurance programs.

Validation of Human Breast Cancer Xenoraft: Immunohistochemical Detection of HA Receptors on Breast Carcinoma

Approximately 8 weeks after tumor induction, two tumor-bearing mice were given a lethal dose of Nembutal. Within 3 minutes of killing the mice, tumors were surgically removed and immediately fixed in 10% v/v buffered formalin for 12 hour. The fixed tumor was dehydrated overnight in a series of 70-100% v/v ethanol, and embedded in paraffin. Sections (2-4 μm) were cut and placed on slides, de-waxed, and brought to water. Slides were washed 3×5 minutes in PBS. Heterophile proteins were blocked by incubation with 10% w/v fetal calf serum for 10 minutes, and then rinsed in PBS. The detection antibodies were applied for 60 minutes at room temperature (RT). The antisera or antibodies were against RHAMM (Applied Bioligands Corporation, Manitoba, Canada), CD44H, CAE and secondary antibodies were purchased from Zymed (California, USA. The slides were washed 3×5 minutes in PBS and endogenous peroxidase activity blocked by immersion in 0.3% H₂O₂ in methanol for 20 minutes. Following a further PBS wash, the peroxidase-conjugated swine anti-rabbit secondary antiserum (Dako, Calif., USA) was applied for 60 minutes at RT, followed by 3×5 minute washes in PBS. Sigma Fast DAB (3,3′-Diaminobenzidine, Sigma, St. Louis, USA) tablets were prepared according to the manufacturer's instructions and the DAB solution was applied for 5-10 minutes at RT. The slides were washed in tap water for 10 minutes, counterstained with hematoxylin, dehydrated and mounted.

Preparation and Injection of CMF/HA Drug Combinations

Cyclophosphamide/methotrexate/5-fluorouracil (CMF) injections were individually made according to mouse masses, to deliver 15 mg/kg MIX/30 mg/kg Cyclo and 30 mg/kg 5-FU, which provides the human equivalent doses of:
15 mg/kg MIX=human equivalent dose 565mg/m²
30 mg/kg Cyclo=human equivalent dose 1 g/m²
30 mg/kg 5-FU=human equivalent dose 400mg/m².

One hundred μl injections were prepared by adding the 5-FU to mtx and drawing the drug combination into a 1 m: syringe the cyclo was then drawn into an individual syringe.

For 5-FU/HA, a pyrogen-free, HA stock solution (10 mg/ml; modal mᵣ 8.5×10⁵ da) was added to a portion of the 20 mg/ml 5-FU stock solution and incubated overnight with vortexing, to a final HA concentration equivalent to 12.5 mg/kg of mouse mass. injections were individually made according to mouse masses, to deliver 30 mg/kg 5-FU and 12.5 mg/kg HA in 100 μl.

HA/CMF injections were individually made according to mouse masses, to deliver 15 mg/kg MTX/30 mg/kg Cyclo/30 mg/kg 5-FU and 12.5 mg/kg HA, which provides the human equivalent doses indicated above.

Adding the mtx to 5-FU/HA and preparing a 100 μL injection prepared the drug combination. the Cyclo was then drawn into an individual syringe.

Mice were randomly divided into the following treatment groups:
Saline (D)ay 1 and Day 2 of a 7 day cycle)
12.5 mg/kg HA 850 kD (Day 1 and Day 2 of a 7 day cycle)
12.5 mg/kg HA 850 kD (Day I and Day 3 of a 7 day cycle)
CMF ()ay 1 and Day 2 of a 7 day cycle)
12.5 mg/kg HA 850 kD/CMF (Day 1 and Day 2 of a 7 day cycle)
CMF (ay 2 and Day 4 of a 7 day cycle)
12.5 mg/kg HA 850 kD on Days 1 and 3; CMF (Days 2 and Day 4 of a 7 day cycle).

The treatments were quantitatively administered via the tail vein. The animals were injected with Cyclo followed, 2 minutes later, by 5-FU/MTX±HA. Animals were weighed and tumor volumes measured on a daily basis.

Collection and Processing of Tumor and Body Organs

At the experimental end-point, when animals had to be euthanased due to degree of disease progression or when the 6-week treatment regimen was completed, the animals were anaesthetised by a 0.1 ml intra-peritoneal injection of Nembutal (60 mg/ml), blood was collected, and the animals killed using cervical dislocation.

Immediately after killing the mouse, the tumor, liver, heart, spleen, bladder, left and right kidneys, uterus, lungs, stomach, intestines, brain and lymph nodes were excised and weighed, and placed in 10% V/V formalin. The tissue was fixed for 16-24 hrs before histological processing. Fixed tissue was dehydrated stepwise to 100% v/v ethanol and embedded in paraffin blocks from which 2-4 μm sections were placed on glass microscope slides. Staining the tissue sections with a hematoxylin nuclear stain and eosin cytoplasmic stain highlighted any pathological features that could indicate treatment toxicity.

Monitoring of Treatment Side-Effects

Gastro-Intestinal (GI) Tract Toxicity

Animals were monitored for GI tract upset, such as diarrhoea, and more severe toxicity manifestations, such as weight loss. Weight loss was monitored by calculating net body weight as estimated by subtracting tumor weight, which was calculated as 1 g×tumor volume (cm³) as cited in Shibamoto et al, Br. J. Cancer 74(11):1709-1713, 1996. For demonstration of any weight changes the animal body weight was normalized to the body weight at the time of treatment commencement as:

$$\frac{\begin{matrix}Body mass ( ex \text{-} tumor ) - \\ body mass at commencement of treatment ( ex \text{-} tumor )\end{matrix}}{Body mass at commencement of treatment ( ex \text{-} tumor )} \times 100$$

Bone Marrow Suppression

Erythirocyte, platelet and white blood cell numbers were estimated by making a 1/50-1/2000 dilution of blood in mouse tenacity saline and counting on a hemocytometer. A blood smear was made and stained with Giemsa, thereby enabling a relative percentage quantification of neutrophils, lymphocytes, and erythrocytes. The total estimation of blood cell sub-populations was compared with published data for mouse blood.

Effect of Treatment on Organ Mass

To ensure that treatments did not induce organ atrophy or enlargement, the organs were removed and weighed during the post-mortem blood. The mass of each organ was calculated as a percentage of the overall net bodyweight, and compared to the organ masses of the saline only group.

Overall Population Survival Time

The overall survival time was calculated as the time (days or weeks) that the animal lived, after the commencement of treatment.

Effect of HA on CMF Induced Toxicity

Gastro-Intestinal Toxicity: Monitoring of Body Mass

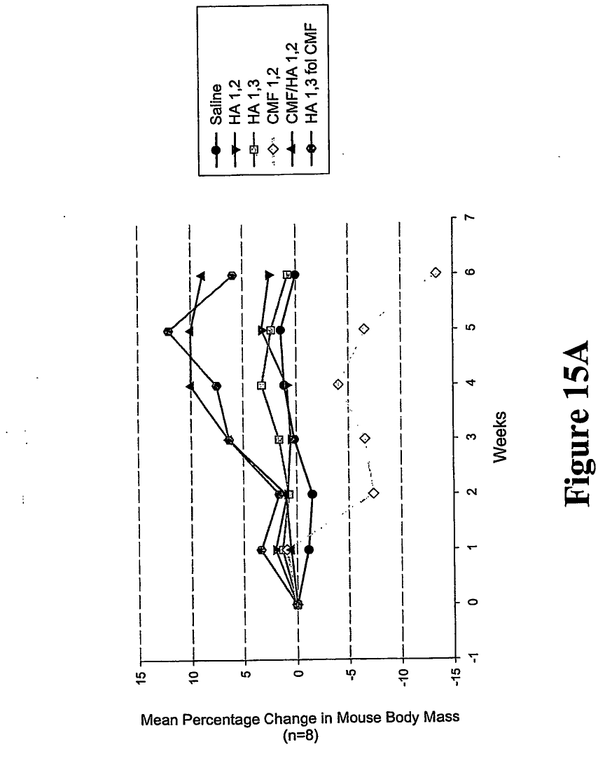

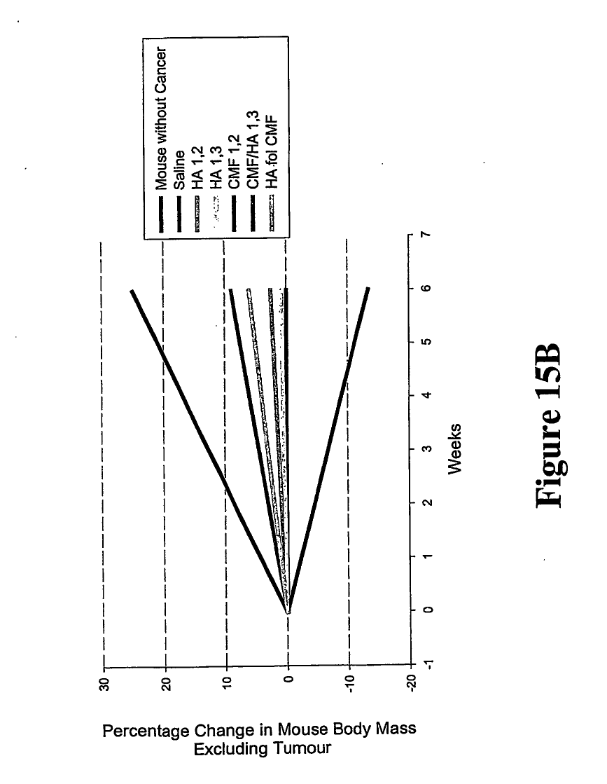

The presence of GI toxicity in the form of diarrhoea was monitored daily and none of the treatment groups displayed symptoms. In addition to visual indications of GI toxicity, net animal body mass (excluding tumor) was also observed for the duration of the six week study. The pre- or co-administration of HA with CMF resulted in a statistically significant increase in body mass, when compared to all of the other treatment groups (see Table 12 and FIG. 15).

TABLE 12
Statistical comparison of the effect of HA
on the gastro-intestinal toxicity of CMF

| | Mean % change in | Statistically significant |
|---|---|---|
| | body mass | difference when compared |
| | (excluding | to HA/CMF treatment |
| Treatment | tumor) ± SEM | group (t-test) |
| HA/CMF | 8.74 ± 1.93 | Not applicable |
| administered | | |
| days 1 and 2 | | |
| Saline | 0.09 ± 1.87 | P = 0.005 |
| HA administered | 2.47 ± 1.05 | P = 0.013 |
| days 1 and 2 | | |
| HA administered | 2.78 ± 1.28 | P = 0.022 |
| days 1 and 3 | | |
| HA Days 1 and 3; | 5.97 ± 1.65 | Not Significant |
| CMF days 2 and 4 | | |
| CMF administered | −15.51 ± 4.15 | P < 0.001 |
| days 1 and 2 | | |

Bone Marrow Toxicity: Neutropenia

The CMF treatment regimen resulted in a reduction in the total circulating white blood cells (WBC) subsequently indicating bone marrow toxicity. The pre- or co-administration of HA/CMF appeared to overcome the toxicity (see Tables 13-15). The WBC sub-population most affected by the CMF was the polymorph cell types, where HA/CMF appeared to result in increased numbers, indicating a possible recruitment of neutrophil progenitors to the circulation.

TABLE 13
Effect of CMF/HA treatment on bone marrow toxicity

| | Total counts of blood cell sub-populations (Mean ± SD) | | | | |
|---|---|---|---|---|---|
| | | | | Neutrophils/ | |
| | | | White | eosinophils/ | Lymphocytes/ |
| | Erthrocytes × | Platelets × | Cells × | basophils | Monocytes |
| Treatment | 10⁶/ul | 10³/ul | 10³/ul | (%) | (%) |
| Saline | 8.71 ± 0.43 | 1290 ± 88 | 6.15 ± 0.19 | 54 ± 3 | 46 ± 3 |
| HA/CMF | 8.99 ± 0.07 | 1323 ± 21 | 6.75 ± 0.12 | 58 ± 2 | 42 ± 2 |
| administered | | | | | |
| days 1 and 2 | | | | | |
| HA | 8.99 ± 0.13 | 1374 ± 7 | 6.15 ± 0.03 | 53 ± 1 | 47 ± 1 |
| administered | | | | | |
| days 1 and 2 | | | | | |
| HA | 8.78 ± 0.25 | 1356 ± 41 | 6.15 ± 0.05 | 53 ± 2 | 47 ± 2 |
| administered | | | | | |
| days 1 and 3 | | | | | |
| HA Days 1 | 8.99 ± 0.05 | 1335 ± 38 | 6.54 ± 0.17 | 56 ± 2 | 44 ± 2 |
| and 3; CMF | | | | | |
| days 2 and 4 | | | | | |
| CMF | 8.90 ± 0.17 | 1295 ± 56 | 5.63 ± 0.32 | 45 ± 3 | 56 ± 3 |
| administered | | | | | |
| days 1 and 2 | | | | | |

TABLE 14
Comparison of blood cells as compared to saline treatment group

| | | | | | HA 1, 3 followed | |
|---|---|---|---|---|---|---|
| Cell Type | CMF | CMF/HA | HA 1, 3 | HA 1, 2 | by CMF 2, 4 | Conclusions |
| Red Blood | NO | NO | NO | NO | NO | |
| Cell | | | | | | |
| White Blood | 8% decrease | 9% increase | NO | NO | 6% increase | Addition of HA overcomes |
| Cells | p = 0.003 | p < 0.001 | | | p < 0.001 | the reduction in WBC |
| | | | | | | observed in the CMF group |
| Eosinophils/ | 13% decrease | 8% increase | NO | NO | NO | Co-administration of HA |
| neutrophils | p < 0.001 | p = 0.007 | | | | increases polymorphic cell numbers |
| Monocytes/ | 20% increase | 10% decrease | NO | NO | NO | |
| lymphocytes | p < 0.001 | p = 0.007 | | | | |
| Platelets | NO | NO | NO | 6% increase | NO | |
| | | | | p = 0.010 | | |

TABLE 15
Comparison of blood cells as compared to HA/CMF treatment group

| | | | | | HA 1, 3 followed | |
|---|---|---|---|---|---|---|
| Cell Type | Saline | CMF | HA 1, 3 | HA 1, 2 | by CMF 2, 4 | Conclusions |
| Red Blood | NO | NO | NO | NO | NO | |
| Cell | | | | | | |
| White Blood | 9% decrease | 17% decrease < 0.001 | 9% decrease | 9% decrease | 3% decrease | Addition of HA overcomes |
| Cells | p < 0.001 | | p < 0.001 | p < 0.001 | p = 0.012 | the reduction in WBC |
| | | | | | | observed in the CMF group |
| Eosinophils/ | 9% decrease | 24% decrease | 9% decrease | 9% decrease | 7% decrease | Co-administration of HA |
| neutrophils | p = 0.007 | p < 0.001 | p < 0.001 | p < 0.001 | p = 0.050 | increases polymorphic cell numbers |
| Monocytes/ | 25% increase | 10% increase | 11% increase | 12% increase | 9% increase | |
| lymphocytes | p < 0.001 | p = 0.007 | p < 0.001 | p < 0.001 | p = 0.050 | |
| Platelets | NO | NO | NO | 3% increase | NO | |
| | | | | p < 0.001 | | |

Effect of Treatment on Organ Mass

HA or the addition of HA to CMF resulted in significant enlargement of the spleen when compared to the untreated control (Table 16); pathological examination of the spleen did not demonstrate any abnormal tissue or cellular pathology. HA also appeared to result in a small decrease in heart mass; once again pathological assessment of the tissue did not demonstrate any abnormal pathology. The CMF treatment regimen appeared to generate a uterine atrophy that was substantially overcome by the co-administration of HA. CMF also caused an enlargement of the GI-tract.

TABLE 16
Comparison of organ mass as compared to saline untreated control organ masses

| | | | | | HA 1, 3 followed | |
|---|---|---|---|---|---|---|
| Organ | CMF/HA | CMF | HA 1, 3 | HA 1, 2 | by CMF 2, 4 | Conclusions |
| Spleen | 101% larger | No | No | No | 197% larger | Addition of HA to CMF results |
| | p < 0.001 | | | | p = 0.007 | in spleen enlargement |
| Heart | 14% smaller | No | 11% smaller | 16% smaller | 13% smaller | Addition of HA results in |
| | p = 0.013 | | p = 0.023 | p = 0.029 | p = 0.023 | a slight decrease in the |
| | | | | | | mass of the heart |
| Kidneys | 10% smaller | No | 8% smaller | No | No | Although a slight difference, HA |
| | p = 0.001 | | p = 0.038 | | | may affect kidney mass |
| Uterus | 49% smaller | 102% smaller | No | No | 97% smaller | CMF results in shrinking of the |
| | p = 0.004 | p = 0.001 | | | p = 0.001 | uterus, where HA reduced the |
| | | | | | | toxicity up to 53% |
| Brain | No | 27% larger | No | No | 22% larger | CMF appears to result in a slight |
| | | p = 0.001 | | | p = 0.01 | brain enlargement which is |
| | | | | | | overcome by coadministration of HA |
| Liver | No | No | No | No | 15% larger | |
| | | | | | p = 0.046- | |
| Bladder | No | No | No | No | No | |
| GI tract | 28% larger | 27% larger | No | No | 19% larger | CMF appears to result in an |
| | p = 0.002 | p = 0.002 | | | p = 0.001 | enlargement of the GI tract |
| Lungs | No | 26% larger | No | No | No | |
| | | p = 0.020 | | | | |
| Stomach | 66% larger | 49% larger | No | 14% smaller | 69% larger | No significant differences |
| | p = 0.006 | p = 0.005 | | p = 0.046 | p = 0.002 | between treatments groups |

Effect of Treatment on Organ Pathology

Pathological assessment of the organs of animals treated with CMF showed that the liver did demonstrate some adverse pathology (Table 17). In the liver, areas of focal necrosis and inflammation were observed in the CMF, HA followed by CMF, saline and HA treatment groups. The greatest degree of focal necrosis and inflammation was observed in the CMF groups that received the drug only, or where animals were pre-treated with HA. The addition of HA totally inhibited the necrosis and inflammation of the liver.

TABLE 17
Comparison of treatment on the adverse pathology observed in the lungs and liver

| | | | | | HA 1, 3 |
|---|---|---|---|---|---|
| | | | | | followed by |
| Organ | Saline | CMF/HA | CMF | HA | CMF 2, 4 |
| Liver | 25% with | No adverse | 87.5% with | 25% with | 87.5% with |
| | areas of focal | pathology | areas of focal | areas of focal | areas of focal |
| | liver necrosis/ | | liver necrosis/ | liver necrosis/ | liver necrosis/ |
| | inflammation | | inflammation | inflammation | inflammation |
| Lungs | No adverse | No adverse | No adverse | 12.5% with | No adverse |
| | pathology | pathology | pathology | oedema and | pathology |
| | | | | macrophage | |
| | | | | infiltrate | |

Effect of Treatment on Survival

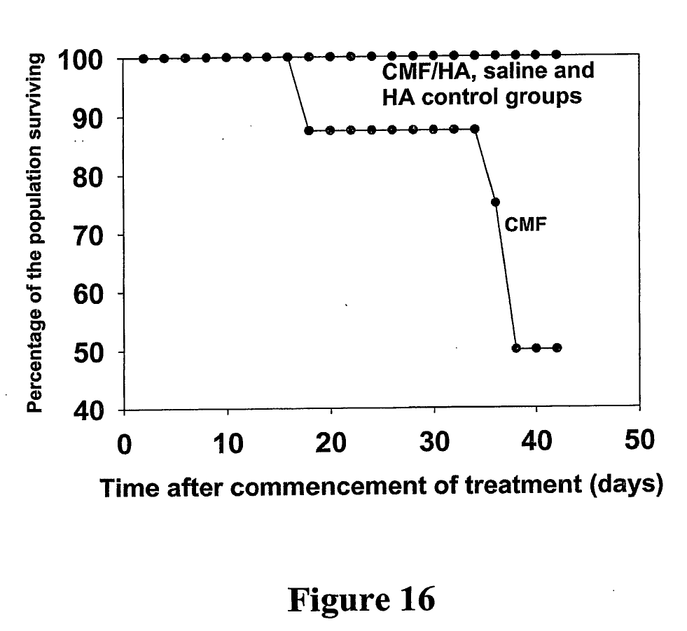

The CMF treatment regimen was very toxic: animals died through excessive weight loss. The other treatment groups did not result in toxicity. The co-administration of HA/CMF totally eliminated treatment toxicity, subsequently resulting in a significantly increased survival period (see Table 18 and FIG. 16).

TABLE 18
Statistical comparison of the effect of HA/CMF on survival

| | | Statistically significant |
|---|---|---|
| | Survival | difference when compared |
| | time (days) | to CMF/HA treatment |
| Treatment | mean ± SEM | group (t-test) |
| HA/CMF administered | 42 ± 0 | Not applicable |
| days 1 and 2 | | |
| Saline | 42 ± 0 | No |
| HA administered | 42 ± 0 | No |
| days 1 and 2 | | |
| HA administered | 42 ± 0 | No |
| days 1 and 3 | | |
| HA Days 1 and 3; | 42 ± 0 | No |
| CMF days 2 and 4 | | |
| CMF administered | 37.38 ± 2.84 | P < 0.05 |
| days 1 and 2 | | |

The foregoing results indicate that the pre- or co-administration of HA with CMF alters the treatment toxicity, while maintaining anti-tumor efficacy.

### EXAMPLE 8

### Effect of Hyaluronan on Doxorubicin Cytotoxicity in H9C2 Rat Embryonic Cardiomyocytes in vitro

H9C2 cardiomyocytes were plated at the density of 50,000 cells/ml/well in DMEM with 10% w/v FCS and allowed to settle overnight in 24well-plates. After 24 hours cells were differentiated for 4 days by growth in 1% w/v FCS. After 4 days, the cells were incubated for 48 hours in growth media containing 0 μg/ml, 0.0097 μg/ml, 0.0195 μg/ml, 0.03905 μg/ml, 0.0781 μg/ml, 0.1562 μg/ml, 0.3125 μg/ml, 0.625 μg/ml, 1.25 μg/ml, 2.5 μg/ml and 5 μg/ml of Dox. Each Dox concentration was done in quadruplets with and without the presence of HA. The molecular weight of HA was 824,000 kD and the concentration used to apply to the cells in presence of Dox was 1 μM, 2.5 μM, 5 μM, 7.5 μM and 10 μM.

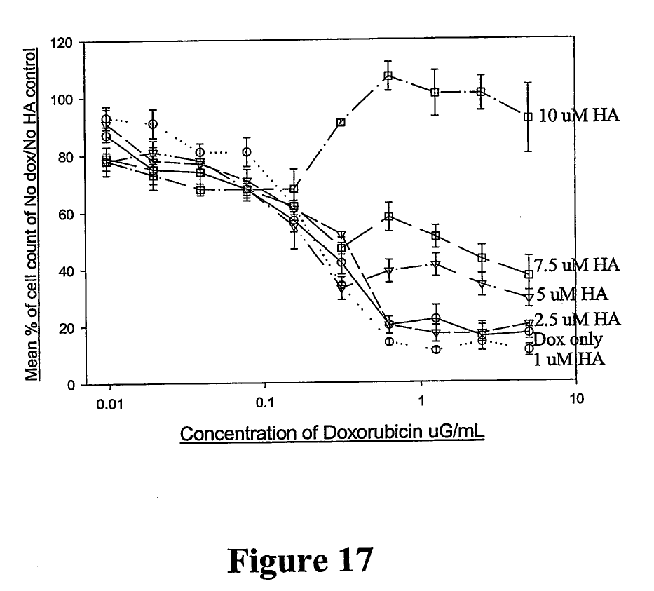

After 48 hours of incubation with Dox±HA, the cells were washed with Hank's, detached by 0.5 ml Trypsin/EDTA to generate a homogenous single-cell suspension and the cells were suspended in a 15 ml of saline. This 15.5-ml cell suspension was used to count the cell number using a Coulter counter. Each concentration of Dox±HA count was expressed as a mean percentage of cell count of No drug/No HA control reading. The results are shown in FIG. 17.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography

Aglietta, M., Piacibello, W., Sanavio, F., Stacchini, A., Apra, F., Schena, M., Mossetti, C., Carnino F, Caligaris-Cappio, F & Gavosto F. Kinetics of human hemopoietic cells after in vivo administration of granulocyte-macrophage colony-stimulating factor. J. Clin. Invest. 83: 551-557, 1989.
Anglin, P., Strauss, B. A., & Brandwein, J. M. Prevention of chemotherapy-induced neutropenia using G-CSF with VACOP-B, A case report. Leuk Lymphoma.11: 469-472, 1993.
Billingham, M. E., Mason, J. W., Bristow, M. R., and Daniels, J. R. Anthracycline cardiomyopathy monitored by morphologic changes. Can. Treat. Rep. 62: 865-872, 1978.
Bodey, G. P., Buckley, M., Sathe, Y. S., Freireich, E. J. Quantitative relationships between circulating leukocytes and infection in patients with acute leukemia. Ann. Intern. Med. 64: 328-340, 1966.
Carter, S. K. Adriamycin-a review. J. Nat. Cancer. Inst. 55: 1265-1274, 1975.
Claiborne, R. A. and Dutt, A. K. Ioniazid-induced pure red cell aplasia. Am. Rev. Respir. Dis. 131(6): 947-949, 1985.
Cohen, A. M., Zsebo, K. M., Inoue, H., Hines, H., Hines, D.,Boone, T. C., Chazin, V. R., Tsai, L., Ritch, T., and Souza, L. M. In vivo stimulation of granupoiesis by recombinant human granulocyte colony-stimulating factor. Proc. Natl. Acad. Sci. 84: 2484-2488, 1987.
Cohn, V. H. and Lyle, J., Anal Biochem.14(3): 434-404, 1966.
Dotti, G., Carlo-Stella, C., Mangoni, L., Cottafavi, L., Caramatti, C., Almici, C., and Rizzoli, V. Granulocyte colony-stimulating factor (G-CSF) prevents dose-limiting neutropenia in lymphoma patients receiving standard dose chemotherapy. Haematologica 80:142-145, 1995.
Grigg, A., Begley, C. G., Juttner, C. A., Szer, J., To L. B., Maher, D., McGrath, K. M., Morstyn, G., Fox, R. M and Sheridan W. P. Effect of peripheral blood progenitor cells mobilized by filgrastim (G-CSF) on platelet recovery after high-dose chemotherapy. Bone Marrow Transplant 11: 23-29, 1993.
Hryniuk, W. M, The importance of dose intensity in the outcome of chemotherapy. In: Advances in Oncology: Hellman S, De Vita V, Rosemberg S, Eds., Philadelphia: J. B. Lippincott; pp. 1121-414, 1988.
Kotake, T., Usami. M., Miki, T., Togashi, M., Akaza, H., Kubota, Y., & Matsumura Y. Effect of recombinant human granulocyte colony stimulating factor (lenograstim) on chemotherapy induced neutropenia in patients with urothelial cancer. Int. J Urol. 6: 61-67, 1999.
Lamszus, K., Jin, L., Fuchs, A., Shi, E., Chowdhury, S., Yao, Y., Polverini, P. J., Laterra. J., Goldberg, I. D. and Rosen, E. M. Scatter factor stimulates tumor growth and tumor angiogenesis in human breast cancers in the mammary fat pads of nude mice. Lab. Invest. 76(3): 39-353, 1997.
Legras, S., Levesque, J. P., Charrad, R., Morimoto, K., Le Bousse, C., Clay, D., Jasmin C & Smadja-Joffe F. CD44-mediated adhesiveness of human hematopoietic progenitors to HA is modulated by cytokines. Blood 89: 1905-1914, 1997.
Lévesque, J. P., Leavesley., D. I., Niutta, S., Vadas, M., & Simmons, P. J. Cytokines increase human haematopoietic cell adhesiveness by activation of very late antigen (VLA)-4 and VLA-5 integrins. J. Exp. Med. 181: 1805-1815, 1995.
Maher D W, Lieschke G J, Green M, Bishop J, Stuart-Harris R, Wolf M, Sheridan W P, Kefford R F, Cebon J, Olver I, et al. Filgrastim in patients with chemotherapy-induced febrile neutropenia. A double-blind, placebo-controlled trial. Ann. Intern. Med. 121: 492-501, 1994.
Mavroudis, D. A., Read, E. J., Molldrem, J., Raptis, A., Plante, M., Carter, C. S, Phang, S., Dunbar, C. E. and Barrett, A. J. T cell-depleted granulocyte colony-stimulating factor (G-CSF) modified allogenic bone marrow transplantation for haematological malignancy improves graft CD34+ cell content but is associated with delayed pancytopenia Bone Marrow Transplant 21: 431-440, 1998.
McCord, J. M. and Fridovich. I. The utility of superoxide dismutase in studying free radical reactions. I. Radicals generated by the interaction of sulfite, dimethyl sulfoxide, and oxygen. J. Biol. Chem. 244(22): 6056-6063, 1969.
Paglia, D. E. and Valentine, W. N. Studies on the quantitative and qualitative characterization of erythrocyte glutathione peroxidase. J. Lab. Clin. Med. 70(1): 158-169, 1967.
Pettengell, R., Gurney, H., Radford, J. A., Dealin, D. P., James, R., Wilkinson, P. M., Kane, K., Bentley, J., and Crowther, D. Granulocyte colony-stimulating factor to prevent dose-limiting neutropenia in non-Hodgiin's lymphoma: a randomized controlled trial. Blood 80(6):430-436, 1992.
Scott, J. E., Cummings, C., Brass, A., and Chen, Y. Secondary and tertiary structures of HA in aqueous solution, investigated by rotary shadowing-electron microscopy and computer simulation. Biochem. J 274: 699-705, 1991.
Shibamoto,Y., Murata, R., Miyauchi, S., Hirohashi, M., Takagi, T., Sasai, K., Shibata. T., Oya, N., and Takahashi, M. Combined effect of clinically relevant doses of emitefur, a new 5-fluorouracil derivative, and radiation in murine tumours. Br. J. Cancer 74(11): 1709-1713, 1996.
Shimamura, M., Kobayashi, Y., You, A., Urabe, A., Okabe, T., Komatsu, Y., Itoh, S., and Takaku, F. Effect of human recombinant granulocyte colony-stimulating factor on hematopoietic injury in mice induced by 5-fluorouracil. Blood 69: 353-355, 1987.
Shimamura, M., Takigawa, T., Urabe, A., Okabe, T., Souza, L. M. and Takaku, F. Synergistic effect of dolichyl phosphate and human recombinant granulocyte colony-stimulating factor on recovery from neutropenia in mice treated with anti-cancer drugs. Exp. Hematol. 16: 681-685, 1988.
Simmons, P. J., Zannettino, A, Gronthos, S., Leavesley, D. Potential adhesion mechanisms for localization of haematopoietic progenitors to bone marrow stoma. Leuk. Lymphoma. 12: 353-363, 1994.
Sheridan, W. P., Begley, C. G., Juttner, C. A., Szer, J., To, L. B., Maher, D., McGrath, K. M., Morstyn, G., and Fox, R. M. Effect of peripheral-blood progenitor cells mobilized by filgrastin (G-CSF) on platelet recovery after high-dose chemotherapy. Lancet 339: 640-644, 1992.
Watanabe, T., Dave, B., Heimann, D. G., Lethaby, E., Kissinger, A and Talmadge, J. E. GM-CSF mobilized peripheral blood CD34+ cells differ from steady-state bone marrow CD34+ cells in adhesion molecule expression. Bone Marrow Transplant 19: 1175-1981, 1997.

1. A method which facilitates the prolonged administration of a dose of chemotherapeutic agent to a subject, wherein said dose is higher than a generally accepted effective dose, said method comprising the pre- and/or co-administration of an effective amount of HA.

2. The method of claim 1 wherein a single dose may be up to 200% higher and/or a cumulative dose may be up to 600% higher than generally accepted effective dose.

3. The method of claim 2 wherein the dose of chemotherapeutic agent is from about 10% to about 150% higher than the generally accepted effective dose.

4. The method of claim 3 wherein the dose of chemotherapeutic agent is from about 35% to about 100% higher than the generally accepted effective dose.

5. The method of of claim 1 wherein HA and the chemotherapeutic agent are simultaneously administered.

6. The method of of claim 1 wherein HA is administered from about 24 hours to about 5 minutes before the chemotherapeutic agent.

7. The method of of claim 1 wherein HA is administered from about 12 hours to about 10 minutes before the chemotherapeutic agent.

8. The method of of claim 1 wherein HA is administered about half an hour before the chemotherapeutic agent.

9. The method of of claim 1 wherein the effective amount of HA is from about 0.5 mg to about 20 mg per kilogram body weight per day.

10. The method of of claim 1 wherein the effective amount of HA is from about 5 mg to about 10 mg per kilogram body weight per day.

11. A method for the prolonged treatment of a subject with a dose of a chemotherapeutic wherein a single dose may be up to 200% higher and/or the cumulative dose may be up to 600% higher than a generally accepted effective dose, said method comprising pre- and/or co-administering an effective amount of HA with said chemotherapeutic agent.

12. The method of claim 12 wherein the dose of chemotherapeutic agent is from about 10% to about 150% higher than the generally accepted effective dose.

13. The method of claim 13 wherein the dose of chemotherapeutic agent is from about 35% to about 100% higher than the generally accepted effective dose.

14. The method of of claim 11 wherein HA and the chemotherapeutic agent are simultaneously administered.

15. The method of of claim 11 wherein HA is administered from about 24 hours to about 5 minutes before the chemotherapeutic agent.

16. The method of of claim 11 wherein HA is administered from about 12 hours to about 10 minutes before the chemotherapeutic agent.

17. The method of of claim 11 wherein HA is administered about half an hour before the chemotherapeutic agent.

18. The method of of claim 11 wherein the effective amount of HA is from about 0.5 mg to about 20 mg per kilogram body weight per day.

19. The method of of claim 11 wherein the effective amount of HA is from about 5 mg to about 10 mg per kilogram body weight per day.
